(19) United States
(12) Patent Application Publication
(10) Pub. No.: US 2014/0329782 A1
(43) Pub. Date: Nov. 6, 2014

(54) COMBINED THERAPEUTIC AGENT

(71) Applicants: Anna Mai Joy Juergens, Niederkassel (DE); Heike Juergens, Niederkassel (DE); Lisa Joy Juergens, Niederkassel (DE)

(72) Inventors: Anna Mai Joy Juergens, Niederkassel (DE); Heike Juergens, Niederkassel (DE); Lisa Joy Juergens, Niederkassel (DE)

(21) Appl. No.: 14/239,610

(22) PCT Filed: Aug. 19, 2012
(86) PCT No.: PCT/EP2012/003525

§ 371 Date: Jun. 25, 2014

(30) Foreign Application Priority Data
Aug. 19, 2011 (DE) 10 2011 111 111.9

(51) Int. Cl. A61K 31/59; A61K 31/167; A61K 31/58

(52) U.S. Cl. CPC A61K 31/59; A61K 31/58; A61K 31/167
USPC 514/164; 514/167

(57) ABSTRACT

The invention relates to a combined therapeutic agent, preferably for use at the prophylactic and/or therapeutic treatment of (A) in particular inflammatory diseases of the respiratory tract, in particular bronchopulmonary diseases, or of (B) in particular inflammatory diseases of the gastrointestinal tract, in particular of the intestine. The combined therapeutic agent comprises at least one vitamin D receptor agonist (VDA) and at least one further active substance, provided that the combined therapeutic agent does not contain any constituent and/or active substance from the group consisting of terpenes, monoterpenes, vasoconstrictors, alpha sympathomimetics and/or the physiologically harmless salts or derivatives thereof.

### CROSS-REFERENCES TO RELATED APPLICATIONS

This application is a National Stage filing of International Application PCT/EP 2012/003525, filed Aug. 19, 2012, claiming priority to German Application No. DE 10 2011 111 111.9 filed Aug. 19, 2011, entitled “COMBINED THERAPEUTIC AGENT”. The subject application claims priority to PCT/EP 2012/003525, and to German Application No. DE 10 2011 111 111.9 and incorporates all by reference herein, in their entirety.

### BACKGROUND OF THE INVENTION

The present invention relates to the medical field of prophylactic and/or therapeutic treatment of in particular, firstly, inflammatory airways diseases and, secondly, inflammatory diseases of the alimentary canal.

In particular, the present invention relates to a combination therapeutic suitable in particular for use in the prophylactic and/or therapeutic treatment of, firstly, in particular inflammatory airways diseases, in particular bronchopulmonary diseases, or of, secondly, in particular inflammatory diseases of the alimentary canal, in particular of the intestine.

Furthermore, the present invention relates to the uses of the combination therapeutic according to the invention or of vitamin D receptor agonist (VDA) in combination with specific further active ingredients, in particular for the aforementioned applications.

The term “airways diseases”, as also used in the context of the present invention, is to be understood to be a general designation referring to all, in particular inflammatory, diseases of the upper and lower respiratory tracts, including acute and chronic medical conditions. Examples of airways diseases of the upper respiratory tract are, for example, inflammations of the paranasal sinuses (e.g., rhinosinusitis), whereas examples of airways diseases of the lower respiratory tract are, for example, asthma, bronchitis and COPD.

The term “bronchopulmonary diseases”, as similarly also used in the context of the present invention, is a generic designation in particular for all inflammatory and noninflammatory diseases of the lower repiratory tract (i.e., the bronchial and pulmonary airways), including in particular asthma, bronchitis and chronic obstructive pulmonary disease (known as “COPD”), and is used synonymously in the context of the present invention with the so-called term “airways diseases of the lower repiratory tract”.

Asthma is a chronic inflammatory disease of the airways with long-term bronchial hypersensitivity or hyperreactivity, inflammation of the bronchi and insufficient bronchial clearance, the consequence of bronchial obstruction being that an asthma attack can occur, with a distinction being made in principle between nonallergic (intrinsic) and allergic (extrinsic) asthma; in addition, both mixed types of allergic and nonallergic asthma and mixed types of asthma and COPD are known. Depending on the severity and the associated disease symptoms, the different asthma disease stages are classified according to the so-called 2006 GINA guidelines (“Global Initiative for Asthma”) from GINA I to IV, where stage 1 refers to intermittent asthma, stage 2 refers to mild persistent asthma, stage 3 refers to moderate persistent asthma and lastly stage 4 refers to severe persistent asthma. By contrast, in the new classification (GINA 2007), asthma control is emphasized, and a distinction is thus made between controlled, partially controlled and uncontrolled asthma.

By contrast, bronchitis refers generally to the inflammation of the bronchi, in particular of the bronchial mucous membrane, with a distinction being made between, firstly, acute bronchitis and, secondly, chronic bronchitis. According to the World Health Organization (WHO), chronic bronchitis is defined as a productive cough on most days over at least three months in two successive years and is among the most frequent chronic diseases altogether (approximately 15 to 25%) with consequently huge relevance from a health economic point of view. In the case of chronic obstructive bronchitis, there is long-term bronchial obstruction which usually develops from chronic bronchitis.

The term chronic obstructive pulmonary disease or COPD, as also used in the context of the present invention, is a collective term for chronic obstructive bronchitis and pulmonary emphysema, the term “obstructive” characterizing the typical feature of long-term bronchial narrowing. In this connection, the different COPD disease stages are classified according to the so-called GOLD guidelines (“Global Initiative for Chronic Obstructive Lung Disease”) from GOLD I to IV depending on the severity and the associated disease symptoms.

When treating bronchopulmonary diseases, in particular airways diseases of the aforementioned type, topical or local, in particular inhaled or inhalable, airways therapeutics are often used when the severity is mild to moderate. These include, for example, inhaled bronchodilators and bronchospasmolytics, such as inhaled beta-2 sympathomimetics, inhaled anticholinergics, inhaled corticosteroids or the like. Because of their only topical or local action, the aforementioned inhaled airways therapeutics often cannot develop the desired therapeutic action, and so mostly relatively high dose quantities need to be delivered in order to achieve the desired therapeutic success, or else, in more severe cases, systemic therapeutics, in particular on the basis of corticosteroids, need to be used as required or even on a long-term basis. Because of the high dose quantities which are required and to be used, undesired adverse effects are additionally observed in many cases. Occasionally, insufficient sensitivity of the aforementioned inhaled airways therapeutics is also observed with respect to the airways diseases to be treated. Lastly, a purely topical, in particular inhalational, therapy of the aforementioned airways diseases is associated in particular with the disadvantage that the use of inhaled airways therapeutics does not always lead to sufficient deposition in the peripheral airways, since the smallest airways, for example the terminal and respiratory bronchioles, generally cannot be reached by an inhalational therapy, in particular when pulmonary function is limited, for example in the case of severe COPD.

Thus, for example, topical or local, in particular inhaled or inhalable, corticosteroids are also used—in addition to a basic therapy with so-called bronchodilators—both in the case of asthma and in the case of COPD when the severity is above a certain level. In addition, in the case of severe asthma corresponding to GINA IV, systemic, in particular peroral, corticosteroids are also additionally used.

Inhalable or inhaled corticosteroids, in particular inhalable or inhaled glucocorticoids, which are also referred to synonymously as “inhalative corticosteroids” or just simply by the acronym “ICS”, are among the most important therapeutics for the topical or local, in particular inhalational, treatment of inflammatory airways diseases, in particular in the case of asthma and COPD. In the case of regular inhalation, the mechanism of action consists in a primarily topical or local deposition in the airways, associated with simultaneously effective anti-inflammation through relatively small steroid quantities.

At the local or topical level, inhalable corticosteroids, in particular glucocorticoids, reduce airways inflammation by inhibiting cytokines and arachidonic acid metabolites (AA metabolites) which are released from activated airways epithelial cells and so-called alveolar macrophages which are distal and line the airways. Depending on the inhaled noxious agent and the genetic disposition, different white blood cells reach the airways through the various aforementioned chemotactic and vasodilatory mediators and cause either eosinophilic cell infiltration in the case of asthma or primarily granulocytic cell infiltration in the case of COPD. Said cell infiltration is known as the most important determinant for the development of airways inflammation in asthma and bronchitis.

However, according to the current international and national therapy guidelines, ICSs are not used in the early stages of airways diseases, but instead in particular only in the case of mild persistent asthma (GINA II) and in the case of moderate to severe COPD (GOLD III and IV), long-term therapy mostly being required. With this acknowledged therapy strategy, the increasingly known adverse effects of ICSs and also insufficient sensitivity of ICSs in the case of COPD are taken into account. For this reason, the therapy with ICSs remains reserved only for moderate and severe COPD, which therapy, however, cannot influence the progress of COPD, but can only influence the reduction of exacerbations.

Naturally, of direct importance to the clinical success of a topical therapy with inhaled corticosteroids, in particular glucocorticoids, is the degree of deposition and in particular also the distribution of steroids in the peripheral airways. But even in the case of optimal use of a very wide variety of different respiratory aids for metered-dose aerosols and powder preparations, the therapeutic success is limited not only by the ability of the patient to inhale optimally, but also, on the contrary, primarily by the inhalation principle because of the insufficient treatment of peripheral airways. Fundamental reasons therefor are thus the insufficient deposition of steroids in the small airways (<10⁻⁸ mol/l) and the correspondingly higher deposition of steroids on the mucous membranes of the mouth and the windpipe. After absorption, relatively small steroid quantities thereby consistently also reach the bloodstream in an undesired manner and cause typical steroid-related adverse effects, for example inhibition of cortisol production, development of osteoporosis, cataract formation, etc. These increasingly known adverse effects therefore hitherto limit also the therapeutic use of ICSs for milder forms of COPD, even though ICSs, depending on the severity of the airways disease, are recommended according to the therapy guidelines at yet higher doses and in combination with further therapeutics including oral glucocorticoids over a period of from two to three weeks. The actual reason therefor can also be attributed to a not always optimal efficiency of action of the therapeutics used.

In addition to the aforementioned therapeutic approaches, use is also made, more or less traditionally, of inhalation-administered essential oils or oil mixtures for a relatively short period of time in order to symptomatically treat, for example, bronchitic complaints and in order to facilitate expectoration in the case of hypersecretion, in particular in the case of colds. However, this is a cause-unrelated therapy which is used in particular only in the case of mild, especially acute airways diseases, but can, at best, be administered in a supportive manner in the case of chronic and in particular severe airways diseases.

Furthermore, the term “diseases of the alimentary canal”, as also used in the context of the present invention, is understood to be a general designation referring to in particular inflammatory diseases of the upper and lower segments of the alimentary canal, in particular of the intestine, including acute and chronic medical conditions. Examples of inflammatory diseases of the intestine are in particular inflammatory bowel diseases (IBD), such as Crohn's disease and ulcerative colitis.

Crohn's disease concerns chronic granulomatous inflammations. The lower small intestine (terminal ileum) and large intestine (colon) are preferentially affected, more rarely the gullet (esophagus). A characteristic of Crohn's disease is its discontinuous nature, with segments of intestinal mucosa being affected; thus, multiple intestinal segments can be affected at the same time, which segments are separated from one another by healthy segments. Typical symptoms of Crohn's disease are abdominal pain and diarrhea. The pain occurs particularly often in the right lower abdomen and often after eating or before defecation. The diarrhea is sometimes bloody. The complaints normally occur in so-called episodes. A episode of this type usually lasts several weeks.

A characteristic of ulcerative colitis (Cu) is inflammation of the rectum and the large intestine. In contrast to Crohn's disease, the inflammation underlying the disease spreads continuously starting from the rectum, i.e., from the anus toward the mouth. The inflammation as such is generally restricted to the intestinal mucosa.

Glucocorticoids are the most important medicaments in the treatment of inflammatory bowel diseases and in particular for the treatment of acute episodes of Crohn's disease. The glucocorticoids administered therapeutically and, primarily, systemically or perorally lead, even in severe cases, to remission in half of all patients. In the case of a mild to moderate episode, a certain improvement can be achieved. However, the therapeutic success is not always sufficient, and serious, in particular systemic, adverse effects often additionally occur.

It is therefore an object of the present invention to at least partly avoid or else to at least attenuate the above-described disadvantages of the prior art.

In particular, the present invention shall provide an improved and/or more efficient therapy for the treatment of airways diseases, in particular bronchopulmonary diseases, or for the treatment of inflammatory intestinal diseases, which shall make it possible not only to increase the therapeutic efficiency, but also to reduce the dose of the active component used.

In particular, the present invention shall allow or achieve an improved efficiency of action or a broader application spectrum of locally or topically, in particular inhalationally, administratable airways therapeutics of the aformentioned type (for example, inhaled corticosteroids, bronchodilators and/or bronchospasmolytics, including sympathomimetics, phosphodiesterase inhibitors, parasympatholytics and/or vagolytics, anticholinergics, etc.) particularly with respect to a therapy for treating airways diseases.

Similarly, the present invention shall allow or achieve an improved efficiency of action or a broader application spectrum of in particular systemically or perorally administratable therapeutics (for example, systemic corticosteroids, etc.) particularly with respect to a therapy for treating intestinal inflammations.

To achieve the abovementioned object, the present invention proposes—in a first aspect of the present invention—a combination therapeutic, in particular a pharmaceutical combination therapeutic, as described herein; further, in particular advantageous, designs of the combination therapeutic according to the invention are the subject matter of the dependent claims referring thereto.

The present invention further provides for, in addition, the uses of a vitamin D receptor agonist (VDA), as defined herein. Further, in particular advantageous, designs of the uses according to the invention in this aspect are the subject matter of the dependent claims referring thereto.

### BRIEF SUMMARY OF THE INVENTION

The present invention is, with respect to both the combination therapeutic according to the invention and the uses according to the invention, characterized in that the combination therapeutic of interest according to the invention does not contain an ingredient and/or active ingredient from the group consisting of terpenes, monoterpenes, vasoconstrictors, alpha sympathomimetics and/or the physiologically safe salts or derivatives thereof. In this connection, it is thus also the case that, in the context of the present invention, those compositions used with the combination therapeutic according to the invention in the context of comedication of the indications or diseases of interest also do not contain, in each case, an ingredient and/or active ingredient from the group consisting of terpenes, monoterpenes, vasoconstrictors, alpha sympathomimetics and/or the physiologically safe salts or derivatives thereof.

It is clear that, hereinafter, special designs, embodiments, advantages and the like which are described below only in relation to one inventive aspect to avoid repetition also apply correspondingly with respect to the other inventive aspects, without express mention being required.

In the case of all below-mentioned relative or percentage weight-based figures, in particular quantities, it has to be additionally noted that they are to be selected by a person skilled in the art in the context of the present invention in such a way that the sum of the particular ingredients, active ingredients, additives and/or excipients or the like always comes to 100% or 100% by weight. This is, however, inherently clear to a person skilled in the art.

Besides, it is possible for a person skilled in the art to deviate, on the basis of the application or depending on the individual case, from the below-listed numbers, ranges or quantities, without departing from the context of the present invention.

In addition, all below-mentioned values or parameters or the like can, in principle, be ascertained or determined using standard or standardized or explicitly specified methods of determination or else using methods of determination or measurement which are familiar per se to a person skilled in the art in this field.

On this basis, the present invention will now be more particularly elucidated.

The applicant has now found that, surprisingly, the above-described object can be achieved by providing, in the context of the present invention, a specific combination therapeutic, as defined below. The combination therapeutic, in particular pharmaceutical combination therapeutic, according to the invention is preferably suitable for use in the prophylactic and/or therapeutic treatment of (A) in particular inflammatory airways diseases, in particular bronchopulmonary diseases, or of (B) in particular inflammatory diseases of the alimentary canal, in particular of the intestine. The combination therapeutic according to the invention is additionally notable for the fact that it comprises at least one vitamin D receptor agonist (VDA), in particular in quantities which are effective, preferably pharmaceutically effective, in each case. In addition, the combination therapeutic according to the invention comprises at least one further active ingredient, the further active ingredient being at least one topical, in particular inhaled, corticosteroid in the case of the prophylactic and/or therapeutic treatment of (A) airways diseases. In the case of the treatment of (B) inflammatory intestinal diseases, the further active ingredient is 5-aminosalicylic acid or the physiologically safe salts or esters thereof and optionially at least one in particular systemic corticosteroid.

In this connection, a mandatory property of the combination therapeutic according to the invention for both cases, (A) and (B), is that the combination therapeutic does not contain an ingredient and/or active ingredient from the group consisting of terpenes, monoterpenes, vasoconstrictors, alpha sympathomimetics and/or the physiologically safe salts or derivatives thereof.

This is because the applicant has found that, completely surprisingly, on the basis of the combination according to the invention with the specific use of a vitamin D receptor agonist (VDA), there is synergistic reinforcement of action with respect to both the steroid and 5-acetylsalicylic acid, and on said basis, a combination therapeutic for the mentioned diseases or indications which is efficient in terms of action is provided, which therapeutic leads to improved therapeutic success and/or to a reduction in dose of the pharmacological active components used and also to a more rapid abatement of the disease. In this connection, another possibility is a further combination, in particular in the context of comedication, of the combination therapeutic according to the invention particularly with so-called guideline therapeutics for the particular disease of interest, it having been found to be possible, completely unexpectedly, in the context of the present invention to significantly improve the efficiency of action of the therapeutics used in each case. This will be elaborated below. In this connection, the combination therapeutic according to the invention has in particular also an anti-inflammatory mechanism of action which is significantly increased by the use of a vitamin D receptor agonist (VDA).

The synergistic effect which, completely surprisingly, was found in the context of the present invention and is evidenced in the following exemplary embodiments and which is associated with the combination therapeutic according to the invention can be attributed in particular—without wishing to be restricted to this theory—to the fact that the expression of the vitamin D receptor is regulated by vitamin D or the vitamin D receptor agonist (VDA) itself. Thus, the vitamin D receptor is downregulated in vitamin D deficiencies, which are often present in the case of the indications of interest, and can be upregulated by vitamin D. In the context of the present invention, the therapeutic delivery of vitamin D or of the vitamin D receptor agonist (VDA) in question thus brings about—without wishing to be restricted to this theory—upregulation of the vitamin D receptor, in particular in the therapeutically mentioned quantities, the upregulation being additionally further reinforced by the further active ingredient or the further active component of the combination therapeutic according to the invention. This is because the further active ingredient, in particular in the form of 5-acetysalicylic acid, leads—similarly without wishing to be restricted to this theory—in particular also to inhibition of so-called COX II metabolites, in particular prostaglandins, which are formed in particular in infections and acute and chronic inflammations and can induce downregulation of vitamin D receptors. Through the inhibition of COX II metabolites, it is thus also possible in this way to upregulate the vitamin D receptor, specifically in particular insofar as its downregulation is prevented. In addition, vitamin D inhibits—similarly without wishing to be restricted to this theory—both the protein MKP1 and p38 MAPK. In this respect, vitamin D also leads to influencing of in particular the MAP kinase signal transduction pathway, which can, inter alia, induce inflammatory processes in the body. The specific active combination of the active components underlying the combination therapeutic according to the invention can also lead at this level to the synergistic effect which was found completely unexpectedly.

The further combination with steroids, in particular corticosteroids, that is provided according to the invention achieves—similarly without wishing to be restricted to this theory—as a result of the steroid administration, reduction of prostaglandin synthesis in addition to upregulation of steroid receptors, which are generally important for immunmodulatory processes, and this has a further positive influence on the reduction of inflammatory processes.

The specific combination of active ingredients thus provides, completely unexpectedly, a synergistic effect with respect to the efficiency of action of the combination therapeutic according to the invention, and this can consequently also lead to a reduction in dose of in particular the corticosteroids used.

Usually, healthy individuals with sufficient exposure to the sun are in principle not dependent on additional intake of vitamin D via food, since previtamin D₃ is produced in the skin starting from the vitamin D precursor substance, viz. 7-dehydroxycholesterol, under the influence of the sun or under UV radiation. The unstable previtamin D₃ gives rise, after about 48 hours, to calcitriol, which represents a physiologically active form of vitamin D₃. Furthermore, lumisterol and tachysterol can be formed starting from 7-dehydroxycholesterol to protect against vitamin D₃ overproduction. However, lack of sun or avoidance of sun and also particular diseases require now and again a food-dependent intake of plant vitamin D₂ or ergocalciferol and/or of animal vitamin D₃ or cholecalciferol.

Since, in the body, the active form of vitamin D is provided only by enzymatic processes, endogenous vitamin D metabolism represents a complex network based on numerous successive metabolic steps. In the context of vitamin D metabolism, the various vitamin D metabolites, in particular bound to the vitamin D-binding protein (VDB), reach the liver or are stored in adipose tissue owing to its lipophilicity. In the liver, vitamin D metabolites are metabolized by 25-hydroxylase (CYP2R1) to form calcifediol (25-hydroxyvitamin D or 25-OH D), which has a relatively high binding affinity for the vitamin D receptor (also referred to synonymously as vitamin D₃ receptor) and has a half life of about 2.5 weeks. In this connection, the conversion rate can be from 0.1 to 0.2%/10⁶ cells/hour. The level of calcifediol corresponds to the sun- and/or diet-related quantity of vitamin D₃ (100 to 200 IU/day) and increases in the summer by on average 12 ng/ml.

In the context of vitamin D metabolism, the active metabolite calcitriol (1,25-dihydroxvitamin D or 1,25-DOH D) is lastly produced in the kidneys and other cells by means of 1-alpha-hydroxylase. Compared to calcifediol, calcitriol has sometimes a greater potential for hypercalcemia. The activity of 1-alpha-hydroxylase and thus the renal formation of calcitriol is stimulated by various hormones, in particular parathyroid hormone, estrogens, calcitonin, growth hormones and prolactin, and also by a drop in the phosphate level, in particular hypophosphatemia. By contrast, calcium and calcitriol themselves inhibit 1-alpha-hydroxylase. Besides the cells of the kidneys, 1-alpha-hydroxylase is also expressed in other cells, such as macrophages or granulomas. However, in contrast to the cells of the kidneys, the other cells are not influenced in terms of their activity by parathyroid hormone, calcium and vitamin D metabolites. By contrast, adverse effects of glucocorticoids, chloroquine or ketoconazole are generally associated with calcitriol reduction. In the case of obesity, there is frequently a deficiency in calcifediol owing to its lipophilicity; nevertheless, it is not possible to reliably differentiate between cause and effect of vitamin D deficiency even in the case of obesity.

Vitamin D receptors (VDR) are expressed by various cells, for example monocytes, macrophages, thrombocytes, lymphocytes and also epithelial cells. Consequently, vitamin D receptors (VDR) occur, inter alia, in the airways and in the intestine. The expression of the vitamin D receptor (VDR) for calcitriol is controlled by calcitriol itself. In particular, an excessive rise in the calcitriol concentration is sometimes associated with an inhibition or reduction of the expression of vitamin D receptors (VDR). If calcitriol is present in a concentration of 10⁻⁷ mol/l, the result is a 9-fold expression of VDR, whereas the presence of a higher calcitriol concentration of 10⁻⁶ mol/l merely leads to a 1.6-fold expression of VDR.

By contrast, vitamin D receptors (VDR) for calcifediol are downregulated neither by calcifediol itself nor by calcitriol, and this has to be taken into account particularly with respect to the therapeutic use of calcitriol and/or calcifediol.

Overall, the role of these receptors is largely unexplored. Inter alia, involvement in increasing intracellular calcium concentration, in phospolipase C activity, in regulating DNA transcription and in RNA synthesis is suspected with a higher binding affinity for calcitriol compared to calcifediol. In various cell models, progressive cell differentiation and inhibition of cell proliferation owing to calcitriol has additionally been described, and there are indications that calcifediol too plays a role here.

The approximate recommended daily requirement for vitamin D or vitamin D₃ is 600 IU and, in the case of elderly patients or in the case of increased risk of osteoporosis, the maximum daily intake is 800 IU and 4000 IU, respectively, in order to achieve a calcifediol level of at least 20 ng/ml. Other recommendations for the treatment of osteoporosis are based on a daily intake of 2000 IU in order to ensure a plasma level of >30 ng/ml. Here, however, there is a lack of proven experience for avoiding the development of various diseases in the event of overdosage, including arteriosclerosis and pancreatic cancer. In this connection, the US Institute of Medicine recommends an oral calcitriol intake of 50 000 IU/week over a period of eight weeks. Subsequently, the calcitriol supply is lowered to 50 000 IU every two to four weeks. Alternatively, the administration of 100 000 IU over a period of three months is mentioned.

Overall, there is a multiplicity of diseases associated with a vitamin D deficiency. These include predominantly chronic diseases, such as various tumor diseases, autoimmune diseases, and also infectious diseases, for example tuberculosis, cardiovascular diseases, such as coronary heart disease and hypertension, Crohn's disease, ulcerative colitis, arthritis, psoriasis and kidney diseases. Treatments with particular therapeutics, in particular corticosteroids and antiepileptics, also lead to a vitamin D or vitamin D₃ deficiency as an adverse effect. Furthermore, mental illnesses, such as depressions or schizophrenia, may also be associated with a vitamin D or vitamin D₃ deficiency. In the case of all the aforementioned diseases, a deficiency of calcitriol can thus generally be detected, although it has so far not been clarified whether the deficiency of vitamin D or vitamin D₃ is the cause or the effect of the disease.

A state of calcitriol deficiency is usually determined by simultaneously determining the precursor or metabolite calcifediol.

It is assumed that—without wishing to be restricted to this theory—the mechanism of action of vitamin D is based on binding of calcifediol or calcitriol to the vitamin D receptor (VDR). This complex binds to the vitamin D response element in the promoter region of the vitamin D response gene, leading to an increase in the RNA polymerase-mediated transcription of the vitamin D response gene. As a result of the reinforced transcription of the vitamin D response gene, various immunologially relevant processes are mediated or induced, including the activation of the CD4 receptors of T helper cells, the inhibition of the T helper cell-produced cytokines (interferon gamma, interleukin 2, interleukin 5), which lead in particular to inflammatory responses, and stimulation of interleukin 4. Furthermore, inhibition of interferon gamma with simultaneous stimulation of interleukin 4, interleukin 5 and interleukin 10 may also occur. Inhibition of the interleukin 12-stimulated production of interferon gamma and also inhibition of interleukin 4 and interleukin 13 by calcitriol may also be effected. It is clear from the above statements that vitamin D, in particular calcitriol, is involved in various immune responses.

Furthermore, it is also assumed that calcifediol might be important for processes of the immune system. In the context of the present invention, it was found that calcifediol and calcitriol inhibit the production of the tumor necrosis factor alpha (TNF-alpha) formed in connection with inflammatory responses, calcifediol inhibiting TNF-alpha formation up to three-times more strongly than calcitriol and thus, surprisingly, bringing about an even stronger anti-inflammation effect. Furthermore, it was found that, surprisingly, calcifediol also interacts synergistically with various guideline therapeutics and reinforces them in terms of their action. In this connection, it is notable in relation to calcitriol that calcitriol in relatively high concentrations sometimes induces downregulation of vitamin D receptors (VDR), and so the action of calcitriol can be attenuated particularly at relatively high concentrations of calcitriol. Nevertheless, in the context of the present invention, calcitriol is also of great significance for the treatment of the diseases of interest.

On this basis, the present invention also proposes comedication of vitamin D or vitamin D metabolites, in particular calcifediol and/or calcitriol, preferably calcifediol, in combination with various anti-inflammatory, bronchodilatory and/or antiallergic guideline therapeutics, as defined below, but with the proviso in each case that the combination therapeutic used for this purpose or the composition used does not contain an ingredient and/or active ingredient from the group consisting of terpenes, monoterpenes, vasoconstrictors, alpha sympathomimetics and/or the physiologically safe salts or derivatives thereof. In particular, comedications based on a combination of, firstly, vitamin D and, secondly, topical or systemic corticosteroids or 5-aminosalicylic acid, as defined herein, with immunosuppressants, long-acting beta-2 sympathomimetics (LABA) or the combination thereof with in particular inhaled corticosteroids, long-acting vagolytics (LAMA) are proposed.

In the context of the present invention, the anti-inflammatory action of vitamin D is of paramount importance. In this regard, of significance according to the invention is, firstly, the anti-inflammatory action of vitamin D per se, which, completely surprisingly, can be increased through a combination with further active ingredients, in particular a monoterpene, in the context of a synergistic, anti-inflammatory effect. In this regard, the present invention provides in particular a type of add-on therapy or comedication for improving the clinical effects or a reduction of medicament consumption even in the context of guideline therapies.

In the case of patients suffering from chronic obstructive pulmonary disease (COPD), a vitamin D deficiency is often present. In this connection, it has been found that the calcifediol level in the blood correlates with the severity of pulmonary function impairment (FEV1) and thus also with the severity of COPD (GOLD stages).

As regards in particular the connection between autoimmune diseases and vitamin D deficiency, it is suspected that, in the case of various autoimmune diseases, a low vitamin D level is caused by a chronic, intracellular infection, which leads to dysfunction of the vitamin D receptor and, in this way, increases in turn the susceptibility to secondary infections. This is suspected particularly for the diseases psoriasis vulgaris, lupus erythematosus, rheumatoid arthritis, sarcoidosis, uveitis and others. Furthermore, patients suffering from chronic polypoid rhinosinusitis and a fungus-allergic rhinosinusitis are in some cases affected by states of vitamin D deficiency, which are also associated with nasal polyps, increased osseous erosion and a reduction of dendritic cells. A vitamin D deficiency also frequently occurs in the case of various forms of inflammatory bowel diseases (IBD), in particular Crohn's disease (CD) and ulcerative colitis (UC).

According to the invention, it was found that, completely surprisingly, the combination therapeutic according to the invention not only makes it possible to achieve an efficient increase in action or reduction in dose of further active ingredients, as used for example in the context of guideline therapies, but can also effectively counter the states of vitamin D deficiency previously found with the diseases of interest, and this represents a further central advantage of the present invention.

However, in the prior art, in connection with vitamin D deficiency diseases which predominantly orginate from observational studies and in some cases from intervention studies in the case of malignant diseases, such as colon cancer, recommendations of national and international associations increasingly state that the vitamin D level should not be determined routinely. In particular, unnecessary vitamin D supplementations are advised against. For instance, the U.S. Institute of Medicine (IOM) recommended, in January 2011, that a vitamin D concentration of 20 ng/ml is entirely sufficient for bone health for 97.5% of the population. Previously, a vitamin D concentration of 30 ng/ml was recommended. Furthermore, it is further argued that “dietary supplements and vitamin D additives to foodstuffs are only meaningful from a medical point of view when there are further risk factors for osteoporosis—for example, in the case of the elderly or in the case of reduced bone density”. Similarly, the Deutsche Gesellschaft für Endokrinologie (German society for endocrinology) also warns that “an oversupply hides health risks and the intake of vitamin D preparations is necessary only in medically justified cases”, in particular against the background of the organism itself forming approximately 80% of the required vitamin D. Proceeding too from the above statements, the specific therapy proposed according to the invention for the mentioned diseases on the basis of vitamin D is not exactly suggested to a person skilled in the art.

This is because, according to the invention, completely surprisingly, specific active effects of vitamin D, in particular calcifediol and/or calcitriol, have now been found, which effects allow the specific uses according to the invention in the context of increases in the efficiency of action and reductions in the dose of accompanying therapeutics or active pharmacological substances.

In this connection, it was also found that, surprisingly, calcifediol, the precursor and metabolite of calcitriol, has, compared to calcitriol or vitamin D in its physiologically active form itself, an action which is primary and more strongly developed and also independent, steroidal and anti-inflammatory, and so calcifediol represents an active component in the form of the vitamin D receptor agonist (VDA) that is particularly preferred according to the invention.

In particular, the anti-inflammatory action of calcifediol exceeds the action of calcitriol by about three-fold, and so calcifediol, as mentioned above, has to be mentioned as one of the preferred substances in the context of the therapeutic use according to the invention, in particular as the combination therapeutic for increasing the action of guideline therapeutics. Nevertheless, with respect to the present invention, calcitriol, however, is also important as vitamin D receptor agonist (VDA).

For the therapeutic use of calcitriol and/or calcifediol or of a mixture of the two aforementioned substances for increasing in particular local, anti-inflammatory vitamin D concentrations and thus the efficacy, the invention provides, in order to ensure optimal availability, a specific therapy concept generally for primary local therapy, on the one hand, for treating airways diseases, in particular in the form of a nasal spray; for inhalational therapy, in particular in the form of a powder and/or metered-dose aerosols; and, on the other hand, for treating inflammatory intestinal diseases (Crohn's disease, ulcerative colitis), in particular in the form of pellets or dissolved or suspended in oil in small intestine-soluble capsules, for local intestinal therapy and systemic therapy in the case of associated systemic inflammation.

### BRIEF DESCRIPTION OF THE DRAWINGS

FIG. 1 provides a graphical representation of the inhibition of the production of TNF-alpha for both calcifediol and calcitriol.

FIG. 2 provides a graphical representation demonstrating the inhibition of the LPS-stimulated TNF-alpha production with increasing concentrations of calcitriol.

FIG. 3 provides a graphical representation demonstrating the inhibition of the LPS-stimulated TNF-alpha production with increasing concentrations of calcitriol, with and without coincubation of calcitriol with formoterol.

FIG. 4 provides a graphical representation demonstrating the inhibition of the LPS-stimulated TNF-alpha production derived from the inhibitory action of budesonide and formoterol in combination in each case with calcitriol compared to the sole use of calcitriol.

FIG. 5 provides a graphical representation demonstrating the inhibition of the LPS-stimulated TNF-alpha production with increasing concentrations of calcitriol with and without coincubation of calcitriol with a plasma-relevant concentration of 5-ASA.

### DETAILED DESCRIPTION OF THE INVENTION

According to the invention, the combination therapeutic for the therapy with vitamin D can thus be provided in a nonlimiting manner in the form of in particular an inhalable solution or suspension based on a powder or spray, in particular associated with the advantage of, independently of body weight and the lipophilicity of the active ingredient, directly treating a local mucous membrane inflammation, e.g., of the airways, on the basis of vitamin D. This approach also avoids any vitamin D-related adverse effects of oral therapy, in particular any development of hypercalcemia.

The present invention thus provides—in a first aspect of the present invention—a combination therapeutic, in particular a pharmaceutical combination therapeutic, preferably for use in the prophylactic and/or therapeutic treatment of (A) in particular inflammatory airways diseases, in particular bronchopulmonary diseases, or of (B) in particular inflammatory diseases of the alimentary canal, in particular of the intestine, wherein the combination therapeutic comprises in particular in quantities which are effective, preferably pharmaceutically effective, in each case—

(a) at least one vitamin D receptor agonist (VDA) and
(b) at least one further active ingredient,
(b1) the further active ingredient being at least one topical, in particular inhaled, corticosteroid in the case of the prophylactic and/or therapeutic treatment of (A) airways diseases or
(b2) the further active ingredient being 5-aminosalicylic acid or the physiologically safe salts or esters thereof and optionally at least one in particular systemic corticosteroid in the case of the prophylactic and/or therapeutic treatment of (B) inflammatory intestinal diseases;
   but with the proviso in each case that the combination therapeutic does not contain an ingredient and/or active ingredient from the group consisting of terpenes, monoterpenes, vasoconstrictors, alpha sympathomimetics and/or the physiologically safe salts or derivatives thereof.

The terms “combination therapeutic” and “pharmaceutical combination therapeutic” or the like, as used in the context of the present invention, are to be understood to be very comprehensive and refer not only to pharmaceutical preparations or pharmaceuticals, but also to so-called medical devices, homeopathic agents, dietary supplements or the like.

Furthermore, the terms “vitamin D receptor agonist” and “vitamin D receptor agonist (VDA)”, as used in the context of the present invention, are to be understood to mean in particular those substances able to form an interaction with the vitamin D receptors of interest, in particular in the manner of a ligand, or to interact with the vitamin D receptors of interest, the interaction of interest leading to activation of the vitamin D receptors in question. In particular, the interaction of the vitamin D receptor agonist used according to the invention with the vitamin D receptors can occur on the basis of binding, in particular on the basis of the so-called “lock-and-key principle”, and as a consequence, activation of the vitamin D receptors of interest can occur with subsequent induction of metabolism-specific processes. Here, it can be provided in particular that the substance as such used according to the invention, i.e., having no further modification or metabolization, is able to interact with the vitamin D receptors. Similarly, the terms “vitamin D receptor agonist” and “vitamin D receptor agonist (VDA)” also encompass those substances which themselves are unable to directly interact with, in particular bind to, the vitamin D receptor, but act to some extent as precursor substances or precursors to provide a ligand for the vitamin D receptor. In this connection, the modification or conversion of the precursors used can occur in particular on the basis of metabolism-specific processes in the body.

In the context of the present invention, the vitamin D receptor agonists used can be in particular substances which are based on synthetic compounds and which correspond to the natural or endogenous metabolites of the endogenous vitamin D metabolism or are at least substantially chemically identical to this end or are identical in terms of action, these being in this respect, as will be defined below, in particular 25-hydroxyvitamin D₃ or 25-hydroxyvitamin D (calcifediol) and/or 1,25-dihydroxyvitamin D₃ or 1,25-dihydroxyvitamin D (calcitriol). In this regard, in the context of the present invention, it is similarly possible in a nonlimiting manner to use the corresponding precursor substances or precursors or storage forms of the substances in question, for example 7-dehydrocholesterol, vitamin D₃ (cholecalciferol) or corresponding inactive storage products, such as lumisterol or tachysterol, even if this is less preferred according to the invention. In particular, the vitamin D receptor agonist can also be used in the form of vitamin D₃ or cholecalciferol as such, which after appropriate delivery or administration—without wishing to be restricted to this theory—is metabolized in the body to the correspondingly active ligands, in particular calcifediol and/or calcitriol.

In the context of the present invention, it is, as already explained, similarly possible to use synthetic analogs of vitamin D₃ and also its precursors as vitamin D receptor agonists. In particular, in the context of the present invention, it is also possible to use vitamin D₃ analogs, such as doxercalciferol, alpha-calciferol, calcipotriol or dihydrotachysterol, as vitamin D receptor agonist.

Similarly, in the context of the present invention, it is also possible to use vitamin D₂ or ergocalciferol and also its analogs.

The vitamin D receptor agonists used according to the invention are in particular substances which can be in the form of so-called secosteroids.

Furthermore, the terms “vitamin D receptor” and “vitamin D receptor (VDR)” used in the context of the present invention refer to in particular ligand-activated transcription factors or receptors which belong to the family of type II steroid receptors and which have in particular an affinity for ligands from the vitamin D group, the receptors in question having in particular a high (binding) affinity for 25-hydroxyvitamin D (calcifediol) and/or 1,25-dihydroxyvitamin D (calcitriol). The receptors in question, which according to the invention act, so to speak, as target structure or target for the ligands used, thus generally represent in particular steroid family-belonging receptors in the form of ligand-activated transcription factors, which are able, as a result of their activation, to activate or to inhibit particular target genes, and can thus influence metabolism in particular also with respect to the regulation of inflammatory processes.

The high binding affinity of the vitamin D receptor for calcifediol or calcitriol also leads to its function, viz. conveying the physiological functions of vitamin D to the biological structures of interest, in particular target cells. Without wishing to be restricted to this theory, vitamin D receptors, after activation by binding of the ligand, form a heterodimer with the so-called retinoid X receptor, the resulting heterodimer serving as a regulatory element for the expression of target genes of the vitamin D receptor. In particular, vitamin D receptors have an important role in the regulation of genes or gene products which are of importance for immune responses, in particular inflammatory processes with prostaglandin release and also for proliferative processes.

In the context of the present invention, it can be provided that (a) the vitamin D receptor agonist (VDA) is selected from the group consisting of compounds of the vitamin D group, in particular vitamin D₃ (cholecalciferol) and/or vitamin D₂ (ergocalciferol), by preference vitamin D₃ (cholecalciferol). In an embodiment particularly preferred according to the invention, (a) the vitamin D receptor agonist (VDA) is the substance 25-hydroxyvitamin D₃ (calcifediol).

Similarly, in the context of the present invention, it can be provided that (a) the vitamin D receptor agonist (VDA) is selected from the group consisting of in particular physiological vitamin D₃ metabolites and/or in particular physiological vitamin D₃ precursors, preferably from the group consisting of in particular physiological vitamin D₃ metabolites. In particular, (a) the vitamin D receptor agonist (VDA) can be selected from the group consisting of in particular synthetic vitamin D₃ analogs, in particular tacalcitol (1,24-dihydroxyvitamin D₃).

According to the invention, particularly good results are obtained with respect to the treatment of in particular inflammatory diseases of the upper respiratory tract or of in particular inflammatory diseases of the intestine when the vitamin D receptor agonist (VDA) is selected from the group consisting of 25-hydroxyvitamin D₃ (calcifediol) and/or 1,25-dihydroxyvitamin D₃ (calcitriol), preferably 25-hydroxyvitamin D₃ (calcifediol).

25-Hydroxyvitamin D₃, which is also referred to synonymously as 25-OH vitamin D₃ or 25-OH D₃ or 25-hydroxycholecalciferol, is in particular a substance of vitamin D metabolism having a ligand function with respect to vitamin D receptors. In the human body, calcifediol, starting from vitamin D₃, can be formed in particular in the liver by hydroxylation of vitamin D₃.

Furthermore, 1,25-dihydroxyvitamin D₃ or calcitriol, also referred to synonymously as 1,25-(OH)₂ vitamin D₃, 1,25-DOH D₃ or 1α,25(OH)₂ cholecalciferol, is a further substance of vitamin D₃ metabolism, it being possible for calcitriol to result from a further hydroxylation of calcifediol.

According to the invention, it is similarly possible for (a) the vitamin D receptor agonist (VDA) to be a combination of 25-hydroxyvitamin D₃ (calcifediol) and 1,25-dihydroxyvitamin D₃ (calcitriol).

In this connection, it has been found to be particularly advantageous when the combination therapeutic comprises (i) 25-hydroxyvitamin D₃ and (ii) 1,25-dihydroxyvitamin D₃ in a weight-based quantity ratio of [(i):(ii)] in the range of 1000:1 to 1:100, in particular in the range of 500:1 to 1:10, by preference in the range of 100:1 to 1:5, preferably in the range of 50:1 to 1:1, particularly preferably in the range of 10:1 to 1:1.

In an embodiment which is further preferred according to the invention, the vitamin D receptor agonist (VDA) can be 25-hydroxyvitamin D₃ (calcifediol).

In general, the quantity of vitamin D receptor agonist (VDA) in the combination therapeutic according to the invention can vary within wide ranges. In particular, in the context of the present invention, it can be provided, in particular in order to ensure sufficient efficacy, that the combination therapeutic according to the invention contains (a) the vitamin D receptor agonist (VDA), in particular 25-hydroxyvitamin D₃ (calcifediol), in a quantity in the range of 0.0001% by weight to 5% by weight, in particular in the range of 0.0005% by weight to 2% by weight, by preference in the range of 0.001% by weight to 1.5% by weight, preferably in the range of 0.005% by weight to 1% by weight, particularly preferably in the range of 0.01% by weight to 0.75% by weight, very particularly preferably in the range of 0.05% by weight to 0.5% by weight, based on the combination therapeutic.

To ensure a high efficacy of the combination therapeutic according to the invention, it can be additionally provided in the context of the present invention that the combination therapeutic contains (a) the vitamin D receptor agonist (VDA), in particular 25-hydroxyvitamin D₃ (calcifediol) and/or 1,25-dihydroxyvitamin D₃ (calcitriol), in a quantity in the range of 0.025 μg to 500 μg, in particular in the range of 0.05 μg to 400 μg, by preference in the range of 0.1 μg to 300 μg, preferably in the range of 1 μg to 200 μg, particularly preferably in the range of 10 μg to 100 μg, based on one dosage and/or administration unit of the combination therapeutic.

In the context of the present invention, it can be additionally provided that the combination therapeutic according to the invention contains at least one physiologically safe carrier (excipient) which is liquid in particular at 20° C. and atmospheric pressure and miscible with (a) the vitamin D receptor agonist (VDA) and/or soluble therein. The carrier or excipient in question is used in particular for the intake of the active substances, preferably on the basis of the vitamin D receptor agonist (VDA) and optionally corticoids used or the like.

In particular with respect to the treatment of (A) in particular inflammatory airways diseases, it can be provided according to the invention that (b1) the topical, in particular inhaled, corticosteroid is a glucocorticoid, preferably selected from the group consisting of beclomethasone, mometasone, budesonide, flunisolide, fluticasone, triamcinolone and the physiologically compatible derivatives, in particular salts and esters, thereof and also mixtures and combinations of the aforementioned compounds.

The corticosteroids are in general a group of approximately 50 steroid hormones formed in the adrenal cortex and also chemically comparable synthetic substances, all corticosteroids arising from the starting material cholesterol and having the progesterone (delta-4-pregnene-3,20-dione) as a common basic structure. The corticosteroids can be divided into three groups according to their biological action and their site of formation, viz. mineralocorticoids, glucocorticoids and androgens. The glucocorticoids preferably used according to the invention thus include the corticosteroids.

To ensure a sufficient efficacy of the combination therapeutic according to the invention, the combination therapeutic should contain (b1) the topical, in particular inhaled, corticosteroid in particular in a quantity in the range of 0.001% by weight to 10% by weight, in particular in the range of 0.01% by weight to 5% by weight, by preference in the range of 0.1% by weight to 2% by weight, based on the combination therapeutic.

The quantity-based ratio between, firstly, (a) vitamin D receptor agonist (VDA) and, secondly, (b1) topical, in particular inhaled, corticosteroid is also of great importance according to the invention: thus, it has been found to be particularly advantageous when the combination therapeutic comprises (a) the vitamin D receptor agonist (VDA) and (b1) the topical, in particular inhaled, corticosteroid in a weight-based quantity ratio of [(a):(b1)] in the range of 100:1 to 1:1000, in particular in the range of 10:1 to 1:500, by preference in the range of 5:1 to 1:100, preferably in the range of 1:1 to 1:50, particularly preferably in the range of 1:1 to 1:10.

As regards, furthermore, the active component used in the case of the treatment of (B) inflammatory intestinal diseases and in the form of (b2) 5-aminosalicylic acid (5-ASA), it can be used in the form of its safe alkali and/or alkaline earth metal salts, preferably in the form of the sodium salt of 5-aminosalicylic acid and/or sodium 5-aminosalicylate.

5-Aminosalicylic acid (also referred to synonymously as 5-ASA or mesalazine) is an amine derivative of salicylic acid having anti-inflammatory action.

According to the invention, it is preferred when the combination therapeutic according to the invention contains (b2) 5-aminosalicylic acid in a quantity in the range of 0.001% by weight to 50% by weight, in particular in the range of 0.01% by weight to 20% by weight, by preference in the range of 0.05% by weight to 10% by weight, preferably in the range of 0.1% by weight to 5% by weight, based on the combination therapeutic.

With respect to a further optimized efficiency of action, the ratio between, firstly, (a) vitamin D receptor agonist (VDA) and, secondly, (b2) 5-aminosalicylic acid in the combination therapeutic according to the invention is also of great importance:

thus, it has been found to be advantageous when the combination therapeutic according to the invention comprises (a) the vitamin D receptor agonist (VDA) and (b2) 5-aminosalicylic acid in a weight-based quantity ratio of [(a):(b2)] in the range of 100:1 to 1:10 000, in particular in the range of 10:1 to 1:5000, by preference in the range of 5:1 to 1:1000, preferably in the range of 1:1 to 1:500, preferably in the range of 1:2 to 1:100.

To further increase or to individually adapt or tailor the efficacy of the combination therapeutic according to the invention with respect to the indication of interest, it can be additionally provided that the combination therapeutic according to the invention comprises at least one further pharmacologically effective ingredient and/or active ingredient. In this respect, the pharmacologically effective ingredient and/or active ingredient can be selected from the group consisting of anti-inflammatory drugs, in particular nonsteroidal anti-inflammatory drugs, preferably acetylsalicylic acid and/or paracetamol; beta sympathomimetics, preferably beta-2 sympathomimetics; parasympatholytics and/or vagolytics; and anticholinergics; and also mixtures and combinations of the aforementioned compounds. In this connection, in particular the nonsteroidal anti-inflammatory drugs are also additionally important—without wishing to be restricted to this theory—in the inhibition of COX II metabolites, in particular prostaglandins, leading to a further increase in the anti-inflammatory efficacy.

A person skilled in the art is capable at any time of selecting the further pharmacologically effective ingredient and/or active ingredient with respect to both its nature and its quantity with regard to the indication or disease of interest. For example, particularly with respect to the treatment of (A) airways diseases, it is possible to use beta sympathomimetics, parasympathomimetics and/or vagolytics and also anticholinergics, whereas with regard to (B) inflammatory intestinal diseases, it is possible to use in particular anti-inflammatory drugs. However, the abovementioned division is not to be understood to be in any way conclusive.

As regards, furthermore, the indication of the (B) inflammatory intestinal diseases, it can be provided in the context of the present invention that (b2) the systemic corticosteroid is a glucocorticoid, in particular a glucocorticosteroid. In this connection, the corticosteroid can be selected from the group consisting of dexamethasone, prednisone, prednisolone, betamethasone, rimexolone, paramethasone, hydrocortisone and the physiologically compatible derivatives, in particular salts and esters, thereof and also mixtures and combinations of the aforementioned compounds.

In this connection, it is advantageous according to the invention when—in particular in order to ensure a sufficient efficiency of action—the combination therapeutic according to the invention contains (b2) the systemic corticosteroid in a quantity in the range of 0.001% by weight to 10% by weight, in particular in the range of 0.01% by weight to 5% by weight, by preference in the range of 0.1% by weight to 2% by weight, based on the combination therapeutic.

According to the invention, the quantity-based ratio between, firstly, (a) vitamin D receptor agonist (VDA) and, secondly, (b2) systemic corticosteroid is also of advantageous importance in the case of the use of a systemic corticosteroid: thus, it can be provided according to the invention that the ratio [(a):(b2)] is in the range of 100:1 to 1:1000, in particular in the range of 10:1 to 1:500, by preference in the range of 5:1 to 1:100, preferably in the range of 1:1 to 1:50, particularly preferably in the range of 1:1 to 1:10.

According to the invention, it can be similarly provided that the combination therapeutic according to the invention contains at least one further vitamin, in particular selected from the group consisting of vitamin A, vitamin C and vitamin E, in particular vitamin C and vitamin E.

In addition, the combination therapeutic according to the invention can comprise at least one further ingredient, in particular selected from the group consisting of processing aids, stabilizers, in particular organic solvents, emulsifiers, antioxidants, humectants, thickeners, antiseptics, dyes, flavorings, odorants, fragrances, diluents, binders, wetting agents, vitamins, trace elements, minerals, micronutrients and/or essential oils and also combinations thereof.

As additionally concerns the combination therapeutic according to the invention, it can be administratable and/or be administered topically. Similarly, the combination therapeutic according to the invention can be administratable and/or be administered systemically, in particular perorally.

In this connection, the topical administration of the combination therapeutic according to the invention, in particular with regard to the use relating to this for the treatment of (A) airways diseases, is a possibility in a nonlimiting manner. In this connection, the term combination therapeutic to be administered topically, in particular inhalationally, is to be understood according to the invention to be very broad, comprising in particular all dosage forms known to a person skilled in the art for treating airways diseases, which dosage forms are suitable for topical, in particular inhalational, use, for example powder-based and/or water-based spray formulations or the like, in particular inhalation and/or throat spray formulations or aerosol sprays for inhalation and/or administration in the throat.

Another possibility is the systemic delivery and/or administration, in particular in peroral form, in particular and in a nonlimiting manner with respect to the treatment of (B) inflammatory diseases of the alimentary canal, in particular of the intestine. In this connection, the combination therapeutic according to the invention can be present or delivered in particular in the form of small intestine-soluble tablets or capsules or else in the form of so-called pellets, leading to high active-ingredient concentrations at the site of action particularly with respect to the treatment of inflammatory intestinal diseases.

A person skilled in the art is capable at any time of selecting and adapting the appropriate dosage or administration form against the background of the disease of interest in each case. For instance, systemic administration is in principle also a possibility for the treatment of in particular inflammatory airways diseases, in particular with respect to systemic intake of the corresponding active components with a correspondingly positive influence or high efficiency of action with regard to both local and systemic (disease) processes.

In general, the combination therapeutic according to the invention can be delivered in daily doses of (a) in the range of 10 μg to 1000 μg, in particular 20 μg to 800 μg, by preference 30 μg to 500 μg, of vitamin D receptor agonist (VDA). In particular, the combination therapeutic can be prepared for delivery in a daily dose of (a) 10 μg to 1000 μg, in particular 20 μg to 800 μg, by preference 30 μg to 500 μg, of vitamin D receptor agonist (VDA).

Similarly—in particular with respect to the treatment of (A) airways diseases—the combination therapeutic can be delivered in daily doses of (b1) in the range of 50 to 1000 μg, in particular 75 to 800 μg, particularly preferably 100 to 600 μg, of topical, in particular inhaled, corticosteroid. In particular, the combination therapeutic can be prepared for delivery in a daily dose of (b1) 50 to 1000 μg, in particular 75 to 800 μg, particularly preferably 100 to 600 μg, of topical, in particular inhaled, corticosteroid.

In addition—in particular with respect to the treatment of (B) inflammatory intestinal diseases—the combination therapeutic can be delivered in daily doses of (b2) in the range of 50 to 10 000 mg, in particular 75 to 6000 mg, particularly preferably 100 to 2000 mg, of 5-aminosalicylic acid. In particular, the combination therapeutic can be prepared for delivery of a daily dose of (b2) 50 to 10 000 mg, in particular 75 to 6000 mg, particularly preferably 100 to 2000 mg, of 5-aminosalicylic acid.

In addition—in particular with respect to the treatment of (B) inflammatory intestinal diseases—the combination therapeutic can be delivered in daily doses of (b2) 50 to 1500 μg, in particular 75 to 1000 μg, particularly preferably 100 to 800 μg, of in particular systemic corticosteroid. In particular, the combination therapeutic can be prepared for delivery in a daily dose of (b2) 50 to 1500 μg, in particular 75 to 1000 μg, particularly preferably 100 to 800 μg, of in particular systemic corticosteroid.

The aforementioned daily doses can be advantageously spread over one, two or more individual administrative doses. In addition, it is at the discretion of a person skilled in the art to deviate from the abovementioned daily doses, in particular with respect to the disease of interest and its severity. However, this is inherently clear to a person skilled in the art.

As regards, furthermore, the combination therapeutic according to the invention, it can, as already explained—in particular with respect to the treatment of (A) airways diseases—be present in a perorally, in particular inhalationally, administratable form, preferably as a suspension and/or in powder form, for example as an inhalational airways spray or throat spray. Alternatively, it can also be provided according to the invention that, as already explained, the combination therapeutic according to the invention is present in a perorally administratable dosage form, in particular in the form of capsules, pellets and/or tablets, by preference in the form of capsules or pellets, preferably capsules. In particular, the combination therapeutic can be present in the form of a peroral, gastric juice-resistant, but small intestine-soluble dosage form, in particular capsules and/or pellets, preferably capsules. The peroral administration is a possibility in particular both in the treatment of (A) airways diseases and in the treatment of (B) inflammatory intestinal diseases, but is preferred in the case of (B) inflammatory intestinal diseases, in particular against the background of obtaining a high active-ingredient concentration at the site of action.

In the context of the present invention, the combination therapeutic according to the invention can be used for the prophylactic and/or therapeutic treatment, in particular combination therapy and/or comedication, of (A) airways diseases, in particular bronchopulmonary diseases, and/or for the prophylactic and/or therapeutic treatment, in particular combination therapy and/or comedication, of (B) in particular inflammatory diseases of the alimentary canal, in particular of the intestine.

In this connection, it can be provided according to the invention that the combination therapeutic according to the invention comprises, firstly, (a) the vitamin D receptor agonist (VDA) and, secondly, (b) the further active ingredient, in particular (b1) the topical, in particular inhaled, corticosteroid or (b2) 5-aminosalicylic acid and/or the systemic corticosteroid, optionally together with the further pharmacologically effective ingredient and/or active ingredient, in particular as already defined, and/or the further vitamin and/or the further ingredient, in a joint and/or single composition. In the context of the present invention, it is possible in this connection for the composition in question to be delivered in turn in the context of comedication with further active ingredients, in particular as presently mentioned.

Alternatively, it can also be provided in a further aspect of the present invention that the combination therapeutic according to the invention, in particular as already defined, preferably for use in the prophylactic and/or therapeutic treatment of (A) airways diseases, comprising—in particular in quantities which are effective, preferably pharmaceutically effective, in each case—firstly, at least one vitamin D receptor agonist (VDA) and, secondly, at least one further active ingredient, the further active ingredient being at least one topical, in particular inhaled, corticosteroid, is in the form of a kit (“kit of parts”) and/or comprises, firstly, (a) the vitamin D receptor agonist (VDA) and, secondly, (b) the further active ingredient, in particular (b1) the topical, in particular inhaled, corticosteroid, in a spatially separated manner, in particular in different compositions, but with the proviso that the combination therapeutic as such does not contain an ingredient and/or active ingredient from the group consisting of terpenes, monoterpenes, vasoconstrictors, alpha sympathomimetics and/or the physiologically safe salts or derivatives thereof.

Also in this respect, the administration forms already described in each case for the respective components are a possibility independently of one another. For example, it can be provided according to the invention that, in a nonlimiting manner, the vitamin D receptor agonist (VDA) is administered in the form of an in particular small intestine-soluble capsule, whereas the (b) further active ingredient is delivered in the form of an inhalational spray composition or the like.

Alternatively, it can be additionally provided in a yet further aspect of the present invention that the combination therapeutic according to the invention, in particular as already defined, preferably for use in the prophylactic and/or therapeutic treatment of (B) inflammatory intestinal diseases, comprising—in particular in quantities which are effective, preferably pharmaceutically effective, in each case—firstly, at least one vitamin D receptor agonist (VDA) and, secondly, at least one further active ingredient, the further active ingredient being 5-aminosalicylic acid or the physiologically safe salts or esters thereof and optionally at least one in particular systemic corticosteroid, is in the form of a kit (“kit of parts”) and/or comprises, firstly, (a) the vitamin D receptor agonist (VDA) and, secondly, (b) the further active ingredient, in particular (b2) 5-aminosalicylic acid or the physiologically safe salts or esters thereof and optionally the systemic corticosteroid, in a spatially separated manner, in particular in different compositions, but with the proviso that the combination therapeutic does not contain an ingredient and/or active ingredient from the group consisting of terpenes, monoterpenes, vasoconstrictors, alpha sympathomimetics and/or the physiologically safe salts or derivatives thereof.

Also in this respect, the administration forms already described in each case for the respective components are a possibility independently of one another. For example, the vitamin D receptor agonist (VDA) on the one hand, optionally in combination with the in particular systemic corticosteroid, can be used in the form of a small intestine-soluble tablet or capsule, whereas the further active ingredient in the form of 5-aminosalicylic acid can be used in the form of a tablet or capsule separate therefrom, i.e. a separate tablet or capsule.

In the context of comedication, for example with corticosteroids, it can be provided according to the invention in a nonlimiting manner that, in particular for the case of (A) the airways diseases, firstly, the vitamin D receptor agonist (VDA), in particular together with the corticosteroid, is delivered locally, in particular by inhalation (=locally administratable components), for example in the form of a spray, and, secondly—together or in combination therewith or else at a different time thereto—at least one further systemically administratable airways therapeutic (=topically administratable component) in particular for the prophylactic and/or therapeutic treatment of airways diseases, in particular bronchopulmonary diseases, is delivered.

According to the invention, it can be provided in particular for the case (D) of the inflammatory intestinal disease that, firstly, the vitamin D receptor agonist (VDA), in particular together with the corticosteroid, is delivered systemically, in particular perorally (=first systemically administratable components), for example in the form of small intestine-soluble capsules, and, secondly—together or in combination therewith or else at a different time thereto—at least one systemically administratable, in particular perorally administered, intestinal therapeutic or 5-aminosalicylic acid (=second systemically administratable component) in particular for the prophylactic and/or therapeutic treatment of in particular inflammatory intestinal diseases is delivered. The first and second systemically administratable components can also be present or delivered in a joint and/or single composition.

However, the embodiments according to the invention are not limiting in any way and a person skilled in the art is capable at any time of selecting the administration form suitable in each case for the particular active component.

The present invention further provides—in a yet further aspect of the present invention—the use of a vitamin D receptor agonist (VDA) for producing a drug for the prophylactic and/or therapeutic treatment of in particular inflammatory airways diseases, in particular bronchopulmonary diseases, wherein the vitamin D receptor agonist (VDA) is delivered and/or used together with at least one topical, in particular inhaled, corticosteroid, but with the proviso that the combination therapeutic does not contain an ingredient and/or active ingredient from the group consisting of terpenes, monoterpenes, vasoconstrictors, alpha sympathomimetics and/or the physiologically safe salts or derivatives thereof.

Similarly, the present invention further provides—in a yet further aspect of the present invention—the use of a vitamin D receptor agonist (VDA) for producing a drug for the prophylactic and/or therapeutic treatment of in particular inflammatory diseases of the alimentary canal, in particular of the intestine, in particular wherein the vitamin D receptor agonist (VDA) is used together with at least one further active ingredient, the further active ingredient being 5-aminosalicylic acid or the physiologically safe salts or esters thereof and optionally at least one in particular systemic corticosteroid in the case of the prophylactic and/or therapeutic treatment of (B) inflammatory intestinal diseases, but with the proviso that the combination therapeutic does not contain an ingredient and/or active ingredient from the group consisting of terpenes, monoterpenes, vasoconstrictors, alpha sympathomimetics and/or the physiologically safe salts or derivatives thereof.

The present invention additionally provides—in an again further aspect of the present invention—the use of a combination therapeutic, in particular as already defined, and at least one further therapeutic for the prophylactic and/or therapeutic treatment, in particular combination therapy and/or comedication, of in particular inflammatory airways diseases, in particular bronchopulmonary diseases, and/or for the prophylactic and/or therapeutic treatment, in particular combination therapy and/or comedication, of in particular inflammatory diseases of the alimentary canal, in particular of the intestine, wherein the combination therapeutic comprises—in particular in quantities which are effective, preferably pharmaceutically effective, in each case—
(a) at least one vitamin D receptor agonist (VDA) and
(b) at least one further active ingredient,
(b1) the further active ingredient being at least one topical, in particular inhaled, corticosteroid in the case of the prophylactic and/or therapeutic treatment of (A) airways diseases or
(b2) the further active ingredient being 5-aminosalicylic acid or the physiologically safe salts or esters thereof and optionally at least one in particular systemic corticosteroid in the case of the prophylactic and/or therapeutic treatment of (B) inflammatory intestinal diseases;
   but with the proviso in each case that the combination therapeutic does not contain an ingredient and/or active ingredient from the group consisting of terpenes, monoterpenes, vasoconstrictors, alpha sympathomimetics and/or the physiologically safe salts or derivatives thereof.

According to the invention, it is possible, as already explained, for the respective active-ingredient components to be present or delivered in an at least partially spatially separated manner and/or to be present or separately delivered spatially separated from the further therapeutic. In this connection, it is in particular also possible for the respective active ingredients or therapeutics to be independently delivered at different times, it being readily possible for the specific design in this respect to be determined by a person skilled in the art with regard to the indication of interest and severity of the disease.

According to the invention, the further therapeutic, in particular in the case of the prophylactic and/or therapeutic treatment of (A) airways diseases, can be a topically administratable airways therapeutic, in particular an inhaled airways therapeutic.

In particular, the topically administratable, in particular inhaled, airways therapeutic can be selected from the group consisting of bronchodilators and bronchospasmolytics. In particular, the topically administratable, in particular inhaled, airways therapeutic can be selected from the group consisting of (i) corticosteroids, in particular glucocorticoids; (ii) sympathomimetics, in particular beta sympathomimetics, preferably beta-2 sympathomimetics; (iii) phosphodiesterase inhibitors; (iv) parasympatholytics and/or vagolytics; (v) anticholinergics; and also mixtures and combinations of the aforementioned compounds, and can be particularly preferably selected from the group consisting of corticosteroids, in particular glucocorticoids, beta-2 sympathomimetics and anticholinergics and also mixtures and combinations thereof.

In an embodiment preferred according to the invention, the topically administratable, in particular inhaled, airways therapeutic can be a corticosteroid, in particular glucocorticoid, preferably selected from the group consisting of beclomethasone, mometasone, budesonide, flunisolide, fluticasone, triamcinolone and the physiologically compatible derivatives, in particular salts and esters, thereof and also mixtures and combinations of the aforementioned compounds. In this connection, the corticosteroid can be delivered in daily doses in the range of 50 to 1000 μg, in particular 75 to 800 μg, particularly preferably 100 to 600 μg. In particular, the combination therapeutic can be prepared for delivery of the corticosteroid in a daily dose of 50 to 1000 μg, in particular 75 to 800 μg, particularly preferably 100 to 600 μg.

In a further embodiment according to the invention, the topically administratable, in particular inhaled, airways therapeutic can be a beta sympathomimetic, preferably a beta-2 sympathomimetic, in particular selected from the group consisting of short-acting beta-2 agonists (SABA), in particular albuterol, fenoterol, hexoprenaline, levalbuterol, metaproterenol, orciprenaline, pirbuterol, reproterol, salbutamol and/or terbutaline, and long-acting beta-2 agonists (LABA), in particular salmeterol and/or formoterol.

The term sympathomimetics indicates those substances which mimic the action of the sympathetic nervous system. Specifically the beta sympathomimetics (also referred to synonymously as “beta agonists”) act predominantly on beta receptors. Very specifically the beta-2 sympathomimetics used according to the invention lead to relaxation on smooth muscles (beta-2 receptors) and have bronchospasmolytic actions. The inhaled beta-2 sympathomimetics preferably used according to the invention can be either short-acting beta-2 agonists (SABA) or long-acting beta-2 agonists (LABA). Examples of short-acting beta-2 agonists (SABA) are in particular albuterol, fenoterol, hexoprenaline, levalbuterol, metaproterenol, orciprenaline, pirbuterol, reproterol, salbutamol and/or terbutaline. Examples of long-acting beta-2 agonists (LABA) are in particular salmeterol and/or formoterol.

In a further embodiment according to the invention, it can be provided that the topically administratable, in particular inhaled, airways therapeutic is an anticholinergic, in particular from the group consisting of ipratropium, tiotropium and/or the physiologically compatible derivatives, preferably salts, thereof, particularly preferably ipratropium bromide and/or tiotropium bromide, and also mixtures and combinations of the aforementioned compounds. Anticholinergics are those substances which suppress the action of acetylcholine, the substances in question similarly having bronchospasmolytic actions.

Similarly, it can be provided according to the invention that at least one further systemic, in particular peroral, active ingredient is administered, in particular selected from the group consisting of systemic phosphodiesterase inhibitors, in particular theophylline; systemic leukotriene receptor antagonists, in particular montelukast, zafirlukast and pranlukast; systemic corticosteroids; anti-inflammatory drugs, in particular nonsteroidal anti-inflammatory drugs, preferably acetylsalicylic acid and/or paracetamol; and also mixtures and combinations thereof.

In the context of the present invention, it can be furthermore provided that the further therapeutic, in particular in the case of the prophylactic and/or therapeutic treatment of (B) inflammatory intestinal diseases, is a systemically administratable therapeutic, in particular a perorally administratable therapeutic, the therapeutic comprising in particular at least one systemically administratable, preferably perorally administratable, corticosteroid, in particular glucocorticosteroid, the glucocorticosteroid being selected in particular from the group consisting of dexamethasone, prednisone, prednisolone, betamethasone, rimexolone, paramethasone, hydrocortisone and the physiologically compatible derivatives, in particular salts and esters, thereof and also mixtures and combinations of the aforementioned compounds.

The delivery of systemic corticoids of this type is a possibility particularly in the context of the treatment of inflammatory diseases of the alimentary canal, in particular of the intestine, but is not restricted thereto, since the delivery of systemic corticoids is in principle also a possibility in the treatment of bronchopulmonary diseases.

In the context of the concept according to the invention, any desired bronchopulmonary diseases or airways diseases can be treated. In particular, the airways disease, in particular the bronchopulmonary disease, is an inflammatory or noninflammatory, in particular inflammatory, disease of the upper or lower respiratory tract. In particular, the bronchopulmonary disease or respiratory disease can be an inflammatory, in particular infection-exacerbated and/or steroid-requiring, airways disease.

For example, the airways disease, in particular bronchopulmonary disease, can be asthma or bronchitis.

In addition, the airways disease, in particular the bronchopulmonary disease, can be a chronic obstructive pulmonary disease (COPD), in particular a chronic obstructive bronchitis or a pulmonary emphysema.

Furthermore, the airways disease, in particular the bronchopulmonary disease, can be a tobacco smoke-induced, in particular nicotine-induced, acute or chronic airways disease, in particular inflammatory airways disease.

In the context of the concept according to the invention, can also be early forms of COPD, in particular in GOLD stage 0 or I, or an early form of asthma, in particular in GINA stage 0 or I. In particular, in the context of the present invention, early forms of COPD, in particular in GOLD stage 0 or I, or early forms of asthma, in particular in GINA stage 0 or I, can be treated, and it is possible in this way to achieve exacerbation prophylaxis before and after exacerbation has occurred, or prevention and slowing down of the disease progress before or after exacerbation has occurred.

Furthermore, the airways disease can be a common cold or an influenzal infection. Similarly, the airways disease can be rhinitis and/or sinusitis.

In an alternative embodiment according to the invention, the inflammatory disease of the alimentary canal, in particular of the intestine, can be Crohn's disease and/or ulcerative colitis and/or an inflammatory bowel disease (IBD).

The combination therapeutic according to the invention is furthermore also suitable for in particular synergistic increasing of the action of at least one topically administratable, in particular inhaled, airways therapeutic in the prophylactic and/or therapeutic treatment of airways diseases, in particular bronchopulmonary diseases.

Furthermore, the combination therapeutic according to the invention is also suitable for in particular synergistic increasing of the anti-inflammatory and/or antioxidative action of topical, in particular inhaled, corticosteroids, in particular glucocorticoids.

Similarly, the combination therapeutic according to the invention is suitable for reducing the dose of topically administratable, in particular inhaled, airways therapeutics, preferably inhaled corticosteroids, in the prophylactic and/or therapeutic treatment of airways diseases, in particular bronchopulmonary diseases.

In a further embodiment according to the invention, the combination therapeutic according to the invention is also suitable for inducing and/or reinforcing the steroid-permissive effect of topically administratable, in particular inhaled, airways therapeutics, preferably inhaled corticosteroids, in the prophylactic and/or therapeutic treatment of airways diseases, in particular bronchopulmonary diseases.

In addition, the combination therapeutic according to the invention is also suitable for avoiding or reducing a habituation effect of beta sympathomimetics in the prophylactic and/or therapeutic treatment of airways diseases, in particular bronchopulmonary diseases, in particular under long-term therapy for all degrees of severity of COPD and asthma.

In addition, the combination therapeutic according to the invention is also suitable in combination with at least one topically administratable, in particular inhaled, airways therapeutic, in particular in combination with an inhaled corticosteroid, for reducing the need for or for replacing systemic corticosteroids or other anti-inflammatory and/or immunosuppressive systemic substances in the prophylactic and/or therapeutic treatment of airways diseases, in particular bronchopulmonary diseases.

In a yet further embodiment according to the invention, the combination therapeutic according to the invention is also suitable in combination with at least one topically administratable, in particular inhaled, airways therapeutic, in particular in combination with an inhaled corticosteroid and optionally an inhaled beta-2 sympathomimetic, for optimizing the basic therapy for asthma and COPD.

In particular, the present invention also provides for the treatment of systemic organ involvement in all severe forms of COPD.

In particular, the combination therapeutic according to the invention is also suitable for modulating and inhibiting COPD-dependent and/or COPD-independent aging processes, in particular with the goal of reducing morbidity, increasing quality of life and/or life expectancy.

In particular, the combination therapeutic according to the invention is also suitable for the combined anti-inflammatory and/or antioxidative therapy of persistent and/or progressive airways diseases, in particular inflammatory airways diseases, after smoking has been given up, optionally under comedication with other airways therapeutics, in particular with the goal of delaying emphysema development, respiratory failure and/or the development of peripheral airways obstructions.

Furthermore, the combination therapeutic according to the invention is also suitable for comedication with in particular topical nasal glucocorticosteroids, in particular for intensifying and/or improving the efficacy and/or for increasing the efficiency of action of guideline therapies for upper respiratory tract diseases, in particular acute, allergic, chronic and/or chronically recurring polypoid rhinitis and/or rhinosinusitis.

In addition, the combination therapeutic is suitable for use in the comedication for intensifying and/or improving the efficacy and/or for increasing the efficiency of action of guideline therapies, in particular on the basis of topical nasal steroids and/or alpha-1 sympathomimetics, for diseases of the upper respiratory tract, in particular acute, allergic and/or chronic rhinitis and/or rhinosinusitis, and/or for use in the comedication for intensifying and/or improving the efficacy and/or for increasing the efficiency of action of guideline therapies, in particular on the basis of LABA, ICS (inhaled corticosteroid), LAMA (long-acting muscarinic antagonist), LABA in combination with ICS, LABA and LAMA, LABA in combination with ICS and LAMA, for diseases of the lower respiratory tract, in particular asthma, COPD and/or chronic pulmonary emphysemas, airways diseases induced by and/or associated with cigarette smoke or fine particles or other environmental noxious agents, in particular with the goal of inducing and/or reinforcing steroid-permissive effects, in particular in order to reduce the progressive course of obstructions and/or emphysemas, exacerbations and/or the need for steroids. In particular, the combination therapeutic can be, in this case, in the form of a small intestine-soluble capsule.

Similarly, the combination therapeutic according to the invention is suitable for use in the comedication and/or as add-on therapy particularly in the form of a kit, having at least one further active ingredient, the active ingredient being selected from the group consisting of systemic glucocorticosteroids (SCS), intravenous replacement therapies with alpha-1 antitrypsin, IgG immunoglobulins, in particular in the case of antibody deficiency syndrome, phosphodiesterase inhibitors, in particular roflumilast and/or theophylline, for the prophylactic and/or therapeutic treatment of diseases of the upper respiratory tract, in particular acute, allergic and/or chronic rhinitis and/or rhinosinusitis, and/or for the prophylactic and/or therapeutic treatment of diseases of the lower respiratory tract, in particular asthma, COPD and/or chronic pulmonary emphysemas, and/or for the prophylactic and/or therapeutic treatment of airways diseases induced by and/or associated with cigarette smoke, fine particles and/or other environmental noxious agents, in particular with the goal of intensifying and/or improving, as already defined, the therapy effects of guideline therapies, and/or with the goal of inducing and/or reinforcing steroid-permissive effects, in particular in order to reduce the progressive course of obstructions and/or emphysemas, exacerbations and/or the need for steroids. In this respect, a replacement therapy with alpha-1 antitrypsin in the case of administration of the vitamin D receptor agonist (VDA) of about 250 mg to 375 mg per week can also be a possibility.

In addition, the combination therapeutic according to the invention is suitable for the compensation of low levels of vitamin D active ingredient, the combination therapeutic being in particular in the form of a kit, for the prophylaxis, therapy and/or delaying of the progressive course of in particular severe inflammatory intestinal diseases, in particular chronically active inflammatory intestinal diseases, and/or for the reduction of exacerbations, of severe COPD, in particular of pulmonary emphysema, possibly with or without respiratory failure and/or severe asthma.

In this connection, the combination therapeutic according to the invention is also suitable for increasing endogenous vitamin D synthesis or for increasing the endogenous level of vitamin D, in particular by oral dosing of small intestine-soluble capsules.

Furthermore, the combination therapeutic according to the invention is also suitable for use in the prophylactic or therapeutic treatment of upper respiratory tract obstructions and also of profibrotic inflammatory responses, in particular in the case of sleep-related breathing disorders, in particular of obstructive and central sleep apnea. Also in this respect, a possibility is in particular small intestine-soluble capsules of the combination therapeutic according to the invention.

Similarly, the combination therapeutic according to the invention is suitable for intensifying and/or improving the efficacy and/or for increasing the efficiency of action of guideline therapies of inflammatory bowel diseases, in particular of irritable bowel syndrome, Crohn's disease and ulcerative colitis, in particular with the goal of prophylaxis, in particular exacerbation prophylaxis, maintenance of remission and increase of anti-inflammation in children and adults, the combination therapeutic being in particular in the form of a kit.

In particular, a focus of the combination therapeutic according to the invention is that of reducing acute, infectious, allergically or chronically related relapse incidences with improvement of therapy refractoriness through combined treatment of local and/or systemic inflammation particularly in the case of irritable bowel syndromes, in particular in the case of inflammatory intestinal diseases, such as inflammatory bowel disease.

In addition, the combination therapeutic according to the invention is suitable for inhalation, in particular in conjunction with ICS, LABA, LAMA, LABA in combination with ICS and also LABA in combination with ICS and LAMA, in particular with the goal of improving the therapy effect of in particular guideline therapies used topically for diseases of the lower respiratory tract. In this connection, the combination therapeutic according to the invention can be in the form of a kit, in particular in the form of a kit for local administration.

In addition, the combination therapeutic according to the invention is also suitable for locally, systemically and/or synergistically reinforcing guideline therapies of inflammatory disease of the alimentary canal, in particular of the intestine, preferably the large and/or small intestine being affected, such as inflammatory bowel diseases (IBD), preferably Crohn's disease and/or ulcerative colitis, in particular with the goal of reducing acute, infectious, allergic and/or chronically related relapse incidences and/or in particular with the goal of improving therapy refractoriness, preferably by combined treatment of local and systemic inflammations, in particular in the case of IBD. In this connection, the combination therapeutic according to the invention should be in the form of a kit, in particular in the form of small intestine-soluble capsules.

The combination therapeutic according to the invention is suitable in particular for reducing the dose of the inhaled corticosteroid in the case of the (A) airways diseases, and/or for reducing the dose of 5-aminosalicylic acid and/or of the in particular systemic corticosteroid in the case of the (B) inflammatory intestinal diseases and/or for reducing the dose of at least one further pharmacological active ingredient and/or ingredient, in particular as already defined, and/or for reducing the dose of at least one systemically or topically administratable pharmacological active ingredient, in particular as already defined, preferably of a corticosteroid. In this connection, the quantity and/or dose of the respective active ingredient to be delivered can be reduced independently, in particular after a defined use period of the combination therapeutic according to the invention, for example after three weeks, in particular after two weeks, by preference after one week, by at least 5%, in particular at least 10%, by preference at least 20%, preferably at least 40%, based on the a corresponding therapeutic having no vitamin D receptor agonist (VDA).

In the context of the concept according to the invention, it is possible to use vitamin D, in particular calcifediol and/or calcitriol, preferably calcifediol, as specific medication in the form of the combination therapeutic defined according to the invention, in particular also in the form of a comedication together with a guideline therapy in various combinations, in particular with the goal of using the independent anti-inflammatory action of vitamin D in combination with various guideline therapeutics, even irrespective of a vitamin D deficiency, in order to reinforce the steroid action of guideline therapeutics, such as SABA, LABA, ICS or LAMA. In particular, it is also possible on this basis to lower the need for steroids or it is possible to provide an alternative to the therapy with steroids.

According to the invention, it is additionally possible to provide in particular the following treatment concepts: treatment of the lower respiratory tract by inhalation of vitamin D and topical corticosteroid with LABA, in particular formoterol; vitamin D and topical corticosteroid, in particular budesonide; vitamin D and topical corticosteroid with LABA and ICS; vitamin D and topical corticosteroid with LAMA; vitamin D and topical corticosteroid with LABA and/or LAMA; vitamin D and topical corticosteroid with further ICS and/or LABA and/or LAMA; in particular also for reinforcing a steroidal anti-inflammatory action, with the goal of reducing steroids, of overcoming steroidal adverse effects and a certain disease-associated steroid resistance.

According to the invention, it is additionally possible to provide in particular the following treatment concepts: systemic treatment of the upper and lower respiratory tracts with vitamin D and topical corticosteroid with montelukast or another leukotriene receptor antagonist or inhibitor; vitamin D with topical corticosteroid with prednisolone; vitamin D with topical corticosteroid with anti-immunoglobulin E (e.g., omalizumab); vitamin D with topical corticosteroid with immunosuppressants; vitamin D and topical corticosteroid with antirheumatics, in particular for reinforcing a steroidal anti-inflammatory action, with the goal of reducing steroids, overcoming steroidal adverse effects and a certain disease-associated steroid resistance.

According to the invention, it is additionally possible to provide in particular additionally the following treatment concepts: achieving an antibiotic and anti-inflammatory action through the combination of vitamin D and corticosteroid with antibiotics.

According to the invention, it is additionally possible to provide in particular also the following treatment concepts: intensifying the anti-inflammatory guideline therapy in the case of inflammatory intestinal diseases on the basis of a combination of vitamin D with 5-ASA; vitamin D with 5-ASA and budesonide.

It is readily possible for a person skilled in the art to identify and realize further designs, modifications and variations and also advantages of the present invention upon reading the description, without departing in these cases from the context of the present invention.

The following exemplary embodiments serve merely to illustrate the present invention without, however, restricting it thereto.

### Exemplary Embodiments and Further Designs Relating to the Present Invention:

Methodology

In the context of the present invention, the influence of the anti-inflammatory action of calcifediol and calcitriol and also, possibly, various further test substances was investigated in vitro in cultures of normal human monocytes. As a measure for determining the anti-inflammatory action, the strength of inhibition of the production of tumor necrosis factor alpha (TNF-alpha), representative of the cytokine production associated with inflammatory processes, was determined in human monocytes. The production of TNF-alpha can be specifically stimulated in human monocytes by the addition of lipopolysaccharides (LPSs). The stronger the anti-inflammatory action of a test substance, the less TNF-alpha produced by the LPS-stimulated monocytes. In the context of the experiments according to the invention, 5-aminosalicylic acid or a corticosteroid was used as further test substance in addition to calcifediol and calcitriol.

In the context of the investigations, human monocytes were firstly isolated from venous blood from donors by standardized methods known in principle to a person skilled in the art (density-gradient centrifugation or magnetic cell separation using MicroBeads®) Subsequently, the purified and highly viable monocytes (95%) were incubated in 48-well culture plates (10⁵/ml) in the presence of the test substances. As controls or comparative experiments, incubations of the monocytes in the presence of calcifediol or calcitriol and also the further test substances in each case alone were carried out. The incubation of the monocytes with calcifediol or calcitriol was used, furthermore, for general detection of the anti-inflammatory action thereof. Furthermore, coincubations of calcitriol or calcifediol in combination with a further test substance were carried out.

During the incubation with the test substances, the production of TNF-alpha (tumor necrosis factor alpha) was stimulated with lipopolysaccharides (10 μg/ml LPS) over a period of 20 hours. Subsequently, the supernatants were obtained and stored at −80° C. until further analysis. For analytical purposes, the various culture supernatants were subjected to an ELISA (enzyme-linked immunosorbent assay), the performance of which is known per se to a person skilled in the art, and in the ELISA the amount of TNF-alpha produced by the monocytes was measured.

The results or investigations shown in detail below report the percentage inhibitory actions of calcifediol or calcitriol, possibly with further test substances, on the production of TNF-alpha by LPS-stimulated human monocytes, in comparison with production of TNF-alpha by monocytes stimulated by LPS, but not incubated in the presence of test substances. The results are shown as percentage inhibition of the LPS-induced control without active ingredient from pooled measurements from at least two to four experiments (n=8 to 12). For statistical analyses, the nonparametric Mann-Whitney test was used, in particular against the background of ascertaining significance in comparison with the LPS control, the sole use of vitamin D₃ or of calcifediol or calcitriol and also the sole use of the test substance.

In the context of the present invention, the inhibitory action is regarded as significant from a p-value of <0.05 with respect to the respective control or the respective comparative assay.

### a) Calcifediol or Calcitriol as Anti-Inflammatory Agent

To detect the anti-inflammatory action of calcifediol or calcitriol, the influence on the

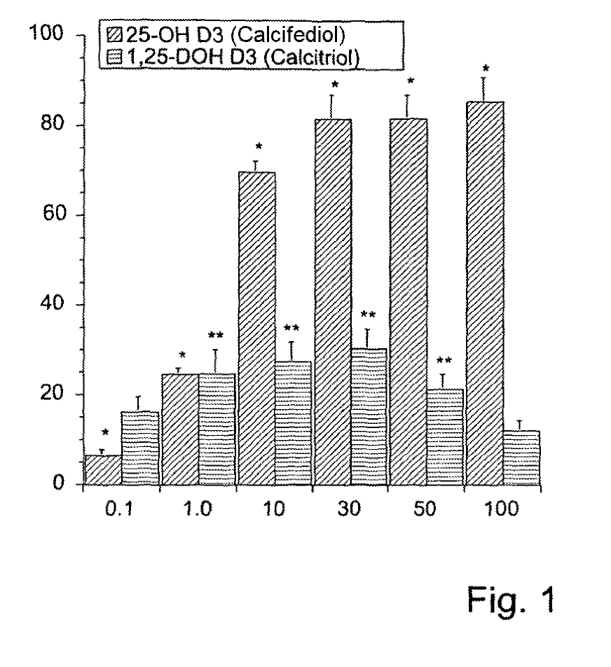

LPS-stimulated production of TNF-alpha by human monocytes was investigated. To this end, calcifediol or calcitriol was used in, in each case, separate experimental series in concentrations of in each case 0.1 ng/ml, 1 ng/ml, 10 ng/ml, 30 ng/ml, 50 ng ml and 100 ng/ml. In this connection, the percentage inhibition of TNF-alpha production by calcitriol or calcifediol is based on the amount of TNF-alpha produced by LPS-stimulated monocytes without the use of inhibitors.
The results show, surprisingly, a significant inhibition of the production of TNF-alpha for both calcifediol and calcitriol, the calcifediol-induced inhibition further exceeding that of calcitriol. The calcifediol-induced inhibition of TNF-alpha varied within the range of −6.5±1% at 0.1 ng/ml to −85.7±5% at 100 ng/ml at a p-value of <0.0117. Thus, the inhibition of the production of TNF-alpha can be considered significant. With calcitriol, it was likewise possible to achieve an inhibition of TNF-alpha, said inhibition, with values within the range of −24.8±5% for 1 ng/ml and −21.4±3% for 50 ng/ml, with a maximal inhibition value of −30.5±4% when using 30 ng/ml calcitriol, turning out to be lower than the calcifediol-induced inhibition. The corresponding results are shown in FIG. 1, the x-axis showing the concentration of calcifediol or calcitriol used and the y-axis showing the respective mean with standard deviation of the percentage inhibition in comparison with the TNF-alpha control (with *p=0.0008 and **p<0.017). FIG. 1 thus shows that, within the therapeutic concentration range of calcifediol (n=8, 2 experiments) and of calcitriol (n=8, 4 experiments), the LPS-stimulated production of TNF-alpha in normal monocytes is significantly inhibited in comparison with the LPS control.
Overall, it was possible to show that vitamin D in its active form, in particular in the form of calcifediol, but also in the form of calcitriol, has an independent anti-inflammatory action. In contrast to calcifediol, the calcitriol-induced inhibition of TNF-alpha falls again slightly at concentrations within the range of 20 to 55 ng/ml. Increasing calcitriol concentrations are thus overall associated with a certain drop in the anti-inflammatory action. Without wishing here to be restricted to this theory, the decreasing anti-inflammation at higher concentrations of calcitriol indicates downregulation of the vitamin D receptor by higher concentrations of calcitriol, and so calcitriol is able to some extent to downregulate its own receptor.
In contrast, the expression of the vitamin D receptor is inducible in situations of vitamin D deficiency and is not adversely affected by supplementation or therapy with calcifediol.
The above results make it possible to conclude that vitamin D, in particular in its active form, is suitable for use in the context of the treatment of non-steroid-requiring, allergic and inflammatory diseases or for steroid reduction, calcifediol being preferred owing to the higher anti-inflammatory action. Specifically, calcifediol is effective in healthy human monocytes even in higher concentrations, and this was hitherto unknown in the prior art.

### b) Vitamin D for Intensifying the Action of Guideline Therapies in the Case of Lower Respiratory Tract Diseases

Background: Previous studies with chronic obstructive pulmonary disease (COPD) provide evidence that the combined inhalational therapy based on inhaled corticosteroids (ICSs), for example budesonide, in combination with LABA, for example formoterol, and also the therapy with LABA and LAMA or else with a selective phosphodiesterase-4 inhibitor (PDE-4 inhibitor) only brings about therapeutic success that is partial and of a delaying nature with regard to the progressive loss of pulmonary function. These substances or substance combinations which are available predominantly inhalationally reduce exacerbations within the range of 14% (LAMA, for example tiotropium) to 25% (ICS, for example budesonide, in combination with LABA, for example formoterol) and lead only to delaying of the progress of the loss in pulmonary function (FEV1) and cannot completely prevent or stop said loss.
An important reason therefor might be that the medication used predominantly inhalationally and thus topically does not sufficiently control the increasingly significant effect of systemic inflammation in COPD. COPD occurs in particular in association with typical vitamin D deficiency diseases, such as osteoporosis, diabetes and cardiovascular diseases. Overall, COPD is relatively difficult to treat.
In comparison, asthma can be treated markedly better, with the unquestionably highly set goal of achieving complete freedom from complaints. An important difference in the case of asthma is the relatively good response to ICS and that there is no indication so far of an association with systemic diseases. However, there are frequently severe allergic and nonallergic accompanying symptoms, mostly also associated with obesity, that require systemic therapy with corticosteroids, and this implies an association with states of vitamin D deficiency.
Overall, it has to be assumed that, both for COPD and for asthma, there is frequently an association with states of vitamin D deficiency.
Approach according to the invention: According to the invention, it has to be assumed in particular that—without wishing to be restricted to this theory—the vitamin D deficiency in COPD and asthma is the primary cause of the varying response to inhalational guideline therapies and thus disease severity. Overall, on the basis of the synergistic interaction of vitamin D in the context of COPD or asthma, it is desirable to achieve an increase in the vitamin D airways concentration, in particular of calcifediol, in order to improve the anti-inflammatory and bronchodilatory action, in particular of ICS in combination with LABA and also LAMA. By contrast—without wishing to be restricted to this theory—any gene-regulatory actions of vitamin D, such as a possible downregulation of disease-specific genes, have at best a fundamental level of importance which is a determining factor for the time of the start of the disease, but not for the severity of the airways disease later on.
According to the invention, it is thus possible for the additional, in particular inhalational, therapy with vitamin D, for example combined with a guideline therapy or alone, to drastically improve the course of airways diseases and, in particular in the case of COPD and constrictive bronchiolitis, to reduce the progress and the exacerbation frequency. In the case of asthma by contrast, it is possible in the context of the therapy concept according to the invention to achieve a bronchodilatory action which significantly reduces the consequences of allergic sensitizations, airways infections and also irritations of the airways by vapors or dusts. Furthermore, it is possible, on the basis of the concept according to the invention, to reduce the therapeutic need for steroids, in particular highly dosed ICSs and systemic corticosteroids.
In this connection, in the context of the present invention, the direct anti-inflammatory action of vitamin D was firstly investigated on isolated monocytes, which express the vitamin D receptor and represent precursors of alveolar macrophages, which line in particular also the lower respiratory tract (FIG. 1).
Subsequently, the anti-inflammatory action of a combination based on vitamin D, in particular calcitriol, and budesonide was investigated. To this end, the inhibitory effect of calcitriol and budesonide was determined both alone and in combination on the LPS-stimulated production of TNF-alpha in human monocytes. The results obtained in this respect can be found in table 1 below.

TABLE 1
Effect of budesonide and calcitriol on the LPS-stimulated
production of TNF-alpha in human monocytes

| | | TNF-alpha | Comparison | |
|---|---|---|---|---|
| | | (pg/10⁵ | with | |
| Conc. | n | monocytes) | control (%) | p-Value |
| No stimulation | 8 | 756.1 ± 192 | — | — |
| 10 μg/ml LPS | 8 | 889.1 ± 52 | — | — |

Calcitriol alone

| | | | | |
|---|---|---|---|---|
| 1 ng/ml | 8 | 712 ± 31 | −20.0 | 0.0117 |
| 10 ng/ml | 8 | 687.5 ± 33 | −22.7 | 0.0008 |
| 30 ng/ml | 8 | 588.8 ± 14 | −33.8 | 0.0008 |
| 50 ng/ml | 8 | 605.6 ± 21 | −31.9 | 0.0008 |

Budesonide alone

| | | | | |
|---|---|---|---|---|
| 10⁻¹⁰ mol | 8 | 606.4 ± 110 | −31.8 | 0.0460 |
| 10⁻⁹ mol | 8 | 412.2 ± 22 | −53.6 | 0.0008 |

Budesonide in combination with calcitriol

| | | | | |
|---|---|---|---|---|
| Bud. 10⁻¹⁰ mol | 8 | 439.4 ± 53 | −50.6 | 0.0008 |
| and Vit. D 1 ng/ml | | | | |
| Bud. 10⁻¹⁰ mol | 8 | 430.0 ± 73 | −51.6 | 0.0008 |
| and Vit. D 10 ng/ml | | | | |
| Bud. 10⁻¹⁰ mol | 8 | 354.4 ± 57 | −60.1 | 0.0008 |
| and Vit. D 30 ng/ml | | | | |
| Bud. 10⁻¹⁰ mol | 8 | 333.4 ± 54 | −62.5 | 0.0008 |
| and Vit. D 50 ng/ml | | | | |
| Bud. 10⁻⁹ mol | 8 | 303.7 ± 12 | −65.5 | 0.0008 |
| and Vit. D 1 ng/ml | | | | |
| Bud. 10⁻⁹ mol | 8 | 266.8 ± 22 | −70.0 | 0.0008 |
| and Vit. D 10 ng/ml | | | | |
| Bud. 10⁻⁹ mol | 8 | 223.1 ± 18 | −73.8 | 0.0008 |
| and Vit. D 30 ng/ml | | | | |
| Bud. 10⁻⁹ mol | 8 | 218.6 ± 27 | −75.5 | 0.0008 |
| and Vit. D 50 ng/ml | | | | |

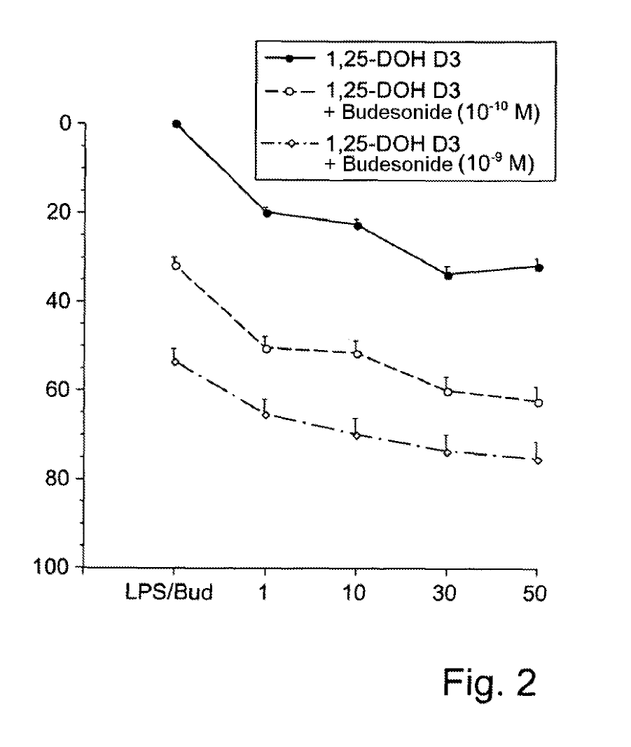

The results listed in table 1 show that, surprisingly, a distinctly stronger inhibition of the production of TNF-alpha in human monocytes can be achieved on the basis of a combination of budesonide and calcitriol than with calcitriol alone or budesonide alone, specifically beyond the action of the individual substances. Thus, in the context of the present invention, a synergistic action between the inhaled corticosteroid budesonide and calcitriol was found, i.e., an increase in the anti-inflammatory action of budesonide in the presence of relevant airways concentrations of calcitriol.
FIG. 2 shows that the LPS-stimulated TNF-alpha production is significantly inhibited by increasing concentrations of calcitriol (solid line). Coincubation of calcitriol with a relatively low airways concentration of budesonide (10⁻¹⁰ mol/l, dashed line) leads to a significant increase in TNF-alpha inhibition in comparison with the control (LPS+budesonide, calcitriol or budesonide alone). These inhibitory effects further increase synergistically after coincubation of calcitriol with a relatively high concentration of budesonide (10⁻⁹ mol/l, dashed-and-dotted line). In FIG. 2, the x-axis shows the concentration of calcitriol used in ng/ml, and the y-axis shows the respective mean with standard deviation of the percentage inhibition in comparison with the TNF-alpha control (with p<0.045 for calcitriol, budesonide, budesonide+calcitriol vs. LPS control; p<0.016 for budesonide 10⁻¹⁰ mol/l+calcitriol vs. calcitriol alone; p=0.0008 for budesonide 10⁻⁹ mol/l+calcitriol vs. calcitriol alone; p<0.0275 for budesonide 10⁻¹⁰ mol/l+calcitriol vs. budesonide 10⁻¹⁰ mol/l alone; p<0.0035 for budesonide 10⁻⁹ mol/l+calcitriol vs. budesonide alone).
Furthermore, the effect of formoterol on the LPS-stimulated TNF-alpha production in normal human monocytes was investigated in vitro. The results obtained in this respect are listed in table 2.

TABLE 2
Effect of formoterol on the LPS- stimulated
production of TNF-alpha in human monocytes

| Conc. of | TNF-alpha | Comparison | |
|---|---|---|---|
| formoterol | (pg/10⁵ | with control | |
| [mol/l] | monocytes) | (%) | p-Value |
| No stimulation | 110.9 ± 72 | — | — |
| 10 μg/ml LPS | 701.6 ± 80 | — | — |
| 10⁻¹² | 693.7 ± 74 | −1.1 ± 11 | 0.8728 |
| 10⁻¹¹ | 653.1 ± 64 | −6.9 ± 10 | 0.3367 |
| 10⁻¹⁰ | 550.9 ± 42 | −21.5 ± 8 | 0.1495 |
| 10⁻⁹ | 491.7 ± 37 | −29.9 ± 7 | 0.1495 |
| 10⁻⁸ | 481.1 ± 21 | −31.4 ± 4 | 0.0163 |
| 10⁻⁷ | 452.9 ± 24 | −35.4 ± 5 | 0.0163 |
| 10⁻⁶ | 487.8 ± 22 | −30.5 ± 4 | 0.0250 |
| 10⁻⁵ | 679.7 ± 34 | −3.1 ± 5 | >0.9999 |

The results listed in table 2 show that formoterol alone at variable concentrations within the range of 10⁻⁹ to 10⁵ mol/l does not have a sufficient inhibitory action on the LPS-stimulated production of TNF-alpha in human monocytes. The p-values ascertained by means of the Mann-Whitney test were above 0.05. Owing to the p-values, no significance can be assumed with respect to the control, i.e., the TNF-alpha production in human monocytes stimulated exclusively with LPS.
Furthermore, in the context of the present invention, it was investigated to what extent a combination on the basis of calcitriol and formoterol can inhibit the LPS-stimulated production of TNF-alpha in human monocytes and thus have an anti-inflammatory action. The results obtained in this respect are listed in table 3.

TABLE 3
Effect of calcitriol (Vit. D) and formoterol (F) on the
LPS-stimulated production of TNF-alpha in human monocytes

| | TNF-alpha | Comparison | |
|---|---|---|---|
| | (pg/10⁵ | with | |
| Conc. | monocytes) | control (%) | p-Value |
| No stimulation | 511.75 ± 198 | — | — |
| 10 μg/ml LPS | 3219 ± 195 | — | — |

Vitamin D₃ alone

| | | | |
|---|---|---|---|
| Vit. D: 1 ng/ml | 2779 ± 254 | −13.9 ± 9 | 0.1333 |
| Vit. D: 10 ng/ml | 2209 ± 245 | −31.4 ± 11 | 0.0179 |
| Vit. D: 30 ng/ml | 2202 ± 263 | −31.6 ± 12 | 0.0209 |
| Vit. D: 50 ng/ml | 2048 ± 256 | −36.4 ± 12 | 0.0079 |

Formoterol alone

| | | | |
|---|---|---|---|
| F: 10⁻⁸ mol/l | 1945 ± 237 | −39.6 ± 12 | 0.0067 |

Calcitriol and formoterol

| | | | |
|---|---|---|---|
| F: 10⁻⁸ mol/l | 1484 ± 196 | −53.9 ± 13 | <0.0001 |
| Vit. D: 1 ng/ml | | | |
| F: 10⁻⁸ mol/l | 1224 ± 176 | −62 ± 14 | <0.0001 |
| Vit. D: 10 ng/ml | | | |
| F: 10⁻⁸ mol/l | 1232 ± 177 | −61.7 ± 14 | <0.0001 |
| Vit. D: 30 ng/ml | | | |
| F: 10⁻⁸ mol/l | 1370 ± 223 | −57.4 ± 16 | <0.0001 |
| Vit. D: 50 ng/ml | | | |

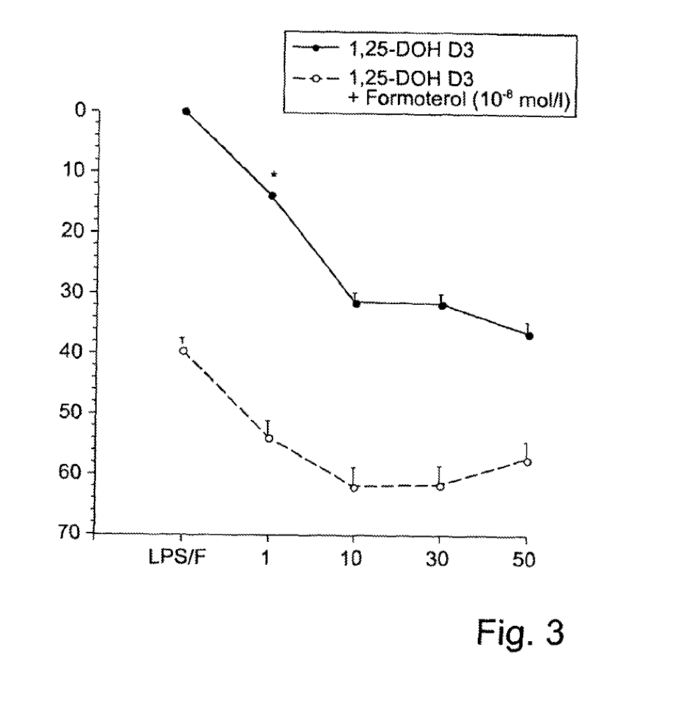

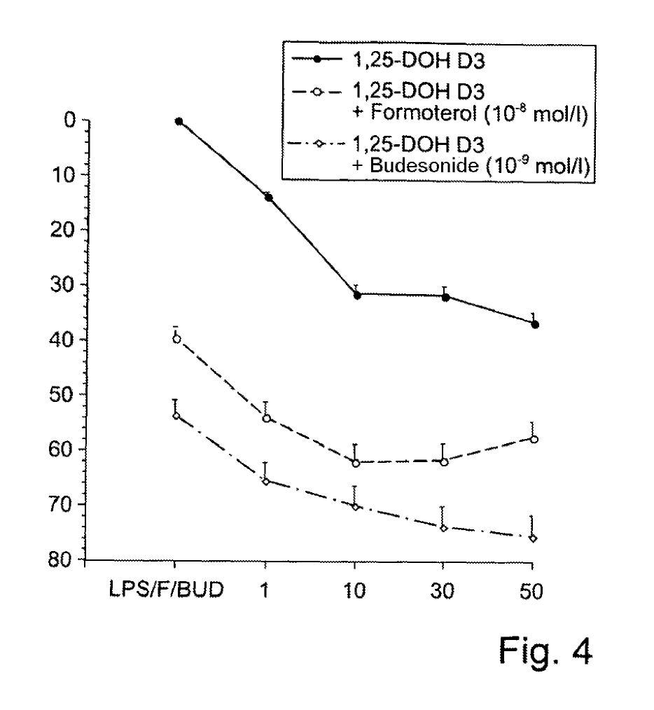

On the basis of the above results in table 3, it is clear that calcitriol and formoterol complement each other synergistically in their action with respect to the inhibition of the LPS-stimulated production of TNF-alpha in human monocytes.
Through the coincubation of the human monocytes in calcitriol and formoterol, it is possible to achieve a significantly stronger inhibition of the TNF-alpha production both in comparison with the control (TNF-alpha production in monocytes by stimulation with LPS) and in comparison with the sole use of calcitriol or formoterol. Thus, a strong anti-inflammatory effect is also to be expected in the case of therapeutic use of a combination based on calcitriol and formoterol. The results listed in table 3 are additionally shown graphically in FIG. 3.
FIG. 3 shows that the LPS-stimulated TNF-alpha production is significantly inhibited by increasing concentrations of calcitriol (solid line). Coincubation of calcitriol with formoterol (10⁻⁸ mol/l, dashed line) leads to a significant increase in the TNF-alpha inhibition in comparison with the control. In FIG. 3, the x-axis shows the concentration of calcitriol used in ng/ml, and the y-axis shows the respective mean with standard deviation of the percentage inhibition in comparison with the TNF-alpha control (with *p>0.1, otherwise p<0.02 for calcitriol, formoterol, formoterol+calcitriol vs. LPS control; p=0.0001 for formoterol vs. calcitriol; p=0.0001 for calcitriol+formoterol vs. calcitriol alone).
FIG. 4 shows, in the context of an overall view of the results shown in tables 1 and 3, the inhibitory action of budesonide and formoterol in combination in each case with calcitriol compared to the sole use of calcitriol. In FIG. 4, the x-axis shows the concentration of calcitriol used in ng/ml, and the y-axis shows the respective mean with standard deviation of the percentage inhibition in comparison with the TNF-alpha control.
Overall, the results listed above are of great significance for the airways therapy in the case of asthma and COPD, especially for the treatment of patients with relatively high severity of disease, in particular obese patients, particularly since there is also a higher degree of systemic inflammation as a function of body weight. In the context of the present invention, it is assumed that—without wishing to be restricted to this theory—the systemic inflammation is caused by a state of vitamin D deficiency during obesity. The invention thus provides, in particular for this difficult patient group, therapy concepts according to the invention that are efficient in terms of action, by means of the combined inhalation with vitamin D.
These results also lead the way because—without wishing to be restricted to this theory—the increasing stimulation of the vitamin D receptor leads to increased expression and thus to an increase in anti-inflammatory, bronchodilatory and exacerbation-protective actions. By contrast, LABA alone leads to downregulation of beta-2 receptors, and so comedication of vitamin D and LABA or else of vitamin D and LAMA leads to reinforcing of the action of guideline therapies. Similarly, in the context of the present invention, a three-fold therapy based on vitamin D, ICS and LABA has been found to be advantageous in comparison with current combined therapy with ICS and LABA.
Overall, in the context of the present invention, it was found that, surprisingly, vitamin D reinforces in particular the action of small topical inhalational concentrations of corticosteroids in terms of their anti-inflammatory efficacy. In particular, the result is also a reinforcement of action in the small airways of the lung periphery, in which the action is typically only insufficient.
In the context of the present invention, the surprising finding was thus a therapeutic use of vitamin D in combination with ICS and/or beta-sympathomimetics particularly in the case of COPD, bronchiolitis and asthma.
Owing to the fact that monocytes produce vitamin D, it can be assumed in the context of the present invention—without wishing to be restricted to this theory—that the comedication of vitamin D, even irrespective of an existing vitamin D deficiency, acts effectively in an anti-inflammatory manner complementarily to the guideline therapy owing to the inhibition of TNF-alpha which is inherent and demonstrated to be reinforced in combination with ICS, and intensifies the guideline effect. In addition, it can be assumed that the comedication of vitamin D in relation to the guideline therapy reduces the increasing severity of airways diseases and is effective in a remission-maintaining manner. As preferred vitamin D, the precursor substance calcifediol is used as comedication with ICS, LABA and LAMA or with an anti-immunoglobulin E (e.g., omalizumab), since the anti-inflammation achieved therewith is stronger in comparison with the use of calcitriol (see FIG. 1).

### c) Vitamin D for Intensifying the Action of the Guideline Therapy in the Case of Inflammatory Intestinal Diseases

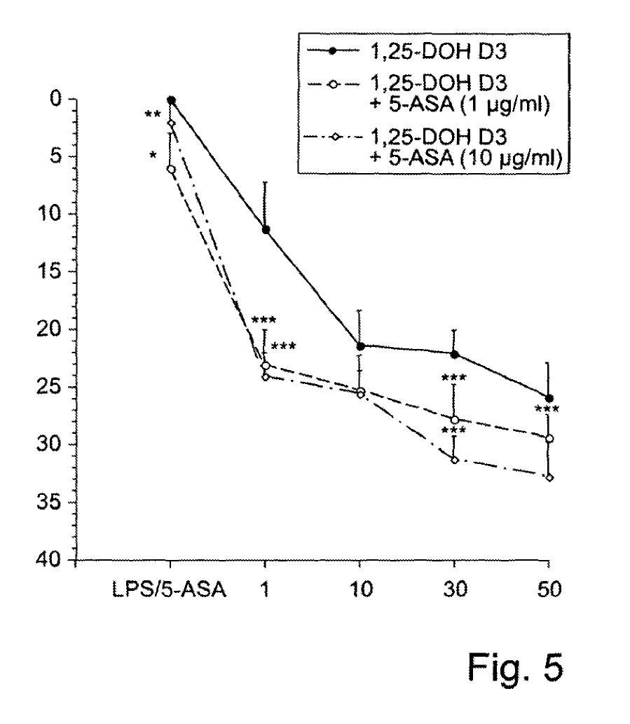

Background: The local therapy in the case of inflammatory intestinal diseases on the basis of a combination of 5-aminosalicylic acid and budesonide is often not sufficient alone and requires additional immunosuppression. Typically, systemically effective azathioprine is used in order to achieve the necessary immunosuppression. The disease progression in these patients with an additional, systemically effective therapy requirement with respect to the local therapy is associated with a high incidence of relapses of about 50% after 3 years owing to the increased inflammation activity and therefore requires in general a long-term therapy.
It is known that budesonide causes only few systemic adverse effects owing to rapid liver metabolism; however, it sometimes mediates insufficient systemic anti-inflammation, but is suitable for treating autoimmune pancreatic diseases. In particular, the use of 6 mg of budesonide in the treatment of Crohn's disease has been found to be ineffective in comparison with the placebo in the mediation of a remission of three months. However, the use of 6 mg of budesonide has been found to be more effective than the administration of 3 mg of mesalazine (5-aminosalicylic acid). Furthermore, it has been shown that the use of 6 mg of budesonide is not more effective than the use of a reduced quantity of 3 mg. On the contrary, frequent systemic adverse effects with cortisol suppression occur when 6 mg of budesonide is delivered. Overall, therefore, budesonide has not been found to be advisable for long-term therapy in the case of Crohn's disease.
As regards the efficacy, the immunosuppressive systemic therapy for maintaining remission with azathioprine (83% remission) is more effective in comparison with local therapy with budesonide (24% remission), but is at the same time also associated with greater adverse effects. Besides the development of a primary sclerosing cholangitis and of articular pain, the role of systemic inflammation in the emergence and progression of an intestinal inflammation is also not considered to be resolved.
In the case of milder forms and for relapse prophylaxis, this is treated to date in a local therapy of the intestine with various small intestine-soluble substances, such as salazosulfapyridine, 5-aminosalicylic acid, para-aminosalicylic acid and locally absorbable glucocorticosteroids, for example budesonide, with a varying, primarily local, anti-inflammatory action. By contrast, in the case of severe forms, it is necessary to use systemically administratable anti-inflammatory substances in the form of generally adverse-effect-abundant immunosuppressants, such as glucocorticosteroids, azathioprine and the like. In the case of children, in particular children under the age of 12, the manifestation of the disease varies and frequently occurs as pancolitis. For approximately 25% of young patients, a colectomy is already necessary in their early years. In addition, the strong adverse effects of the medicaments and also systemic inflammations frequently lead to growth impairments.
A person skilled in the art is aware of 5-aminosalicylates (5-ASA) as main therapeutics for treating mild to moderate and in particular left-sided ulcerative colitis and also for maintaining remission. In comparison to budesonide, they respond distinctly better in the context of the therapy. By contrast, in the case of severe forms of ulcerative colitis, in the case of the small intestine (ileum) and the right-sided colon being affected, in the case of mild to moderate disease activities and also in the case of Crohn's disease, the therapy with steroids, in particular budesonide, is only advisable over a limited period. The systemic use of steroids for treating acute exacerbations is also only advisable over a limited period.
Thus, in the prior art, there is further a need for a highly tolerable and effective, anti-inflammatory therapy concept for the prophylactic maintenance of remission of inflammatory bowel diseases. In principle, an early therapy start is to be aimed at to prevent escalation of the inflammation and a high steroid-equivalence requirement with adverse-effect-abundant immunosuppressants, in order to positively influence the disease course. To achieve this goal, in particular with regard to a therapy start at a very early time in the disease, the invention provides a highly tolerable and effective therapy concept which is geared toward reinforcing the local action of therapeutics used to date, in particular 5-aminosalicylic acid (5-ASA) and budesonide, and has furthermore a highly tolerable systemic efficacy.
Approach according to the invention: Tumor necrosis factor (TNF-alpha) plays a central role in the mediation of cytokine induction and thus of the inflammatory response in various diseases. Therefore, in the investigations undertaken in vitro here, TNF-alpha was used as a measure of the anti-inflammatory action of a substance or substance combination. In the case of ulcerative colitis and Crohn's disease, the cytokine TNF-alpha is formed increasingly by so-called Schwann cells. In comparison with inactive ulcerative colitis or normal cells, both the production and the receptor expression of TNF-alpha is increased in the case of active ulcerative colitis. In the case of patients suffering from inflammatory bowel diseases, in particular Crohn's disease, the expression of relevant type 1 T helper cells is distinctive, leading in particular to an increased release of cytokines, in particular interferon gamma, TNF-alpha and interleukin 2. However, in the case of ulcerative colitis, interleukin 10 may have a compensatory, anti-inflammatory action as antagonist of cytokine production.
With respect to the question of an immunomodulatory importance of the active form of vitamin D, in particular calcifediol and/or calcitriol, 100 μg/ml calcitriol was used to compare effects on the production by type 1 and type 2 T helper cells between 8 patients suffering from Crohn's disease and 9 patients suffering from ulcerative colitis and 6 healthy test subjects in the context of an ex vivo study. To this end, peripheral mononuclear cells isolated from the blood of the test subjects were investigated after an incubation with 100 μg/ml calcitriol.
The results in this respect show that the production of TNF-alpha was significantly increased in the peripheral mononuclear cells isolated from patients suffering from inflammatory bowel diseases in comparison with those of the healthy control group, but could be lowered by incubation of the cells with calcitriol. In addition, it is shown that calcitriol reduces the production of interferon gamma and stimulates the production of interleukin 10. The effect of calcitriol on the interleukin 10 production was not significantly greater in patients suffering from ulcerative colitis than in patients suffering from Crohn's disease. The production of TNF-alpha was inhibited by calcitriol only in cells from patients suffering from Crohn's disease and not in cells from healthy patients. For Crohn's disease and ulcerative colitis, a varying response to vitamin D or its precursors or precursor substances is thus altogether postulated.
To investigate the role of interleukin 10-producing dendritic cells or the consequences of a deficiency of said cells in inflammatory bowel diseases, short-term and long-term incubations of interleukin 10 and dentritic cells with dexamethasone and calcitriol were carried out. In this connection, the production of the CD4 receptor and lymphocytes in comparision with a control was investigated in particular. It is clear from the results obtained in this respect that immunosuppressively acting dendritic cells can protect against an experimentally mediated inflammatory bowel disease. In connection with the new vitamin D receptor agonist calcitriol, it has additionally emerged that the mRNA for TNF-alpha and other cytokines has a stronger inhibitory effect than calcitriol and furthermore acts less hypercalcemically.
In summary, the current situation shows that, to date, calcitriol is decisively known only for the clinical diagnosis of a state of vitamin D deficiency. The causes of states of vitamin D deficiency are fundamentally not yet completely understood; however, there are suspicions that lifestyle might also be important in the case of Crohn's disease, in particular with respect to physiological aspects in terms of nutrition. In this connection, especially the dependence of the vitamin D level on the supply of fish oil having a high proportion of omega-3 fatty acids, prebiotics and oxidants is considered to be relevant with regard to Crohn's disease.
In fundamental contrast to the prior art, surprisingly, a strong anti-inflammatory action for calcifediol was found in the context of the present invention, which action exceeds that of calcitriol by more than three times and furthermore does not decrease again with increasing concentrations. In the context of the present invention, it was thus found that, surprisingly, not only calcitriol but also in particular calcifediol is suitable for the long-term therapy of various inflammatory diseases, in particular inflammatory bowel diseases.
On the basis of the problems of the prior art already outlined, there is an important need to improve the clinically insufficient anti-inflammatory action of 5-ASA for the therapy of even severe forms of ulcerative colitis and also the development of resistance, including against corticosteroids, in particular in the case of Crohn's disease.
Therefore, it was investigated in the context of the present invention whether the anti-inflammatory effect of 5-ASA can be reinforced by vitamin D through the use of a combination of vitamin D and also 5-ASA and any proinflammatory effects of 5-ASA can be reduced by a combination with vitamin D. On this basis, it might be possible to increase the efficacy and tolerability of the standard therapy with 5-ASA.
To this end, the anti-inflammatory effects of a combination of vitamin D and 5-ASA were investigated in vitro in normal human monocytes for the first time in the context of the present invention. The results obtained according to the invention show that 5-ASA in therapeutic concentrations of from 0.040 to 0.12 mg/mg in tissue and from 0.1 to 1.5 mg/ml in plasma significantly inhibits the LPS-stimulated production of TNF-alpha at a tissue-relevant concentration of 0.1 mg/ml and a plasma-relevant concentration of 1 mg/ml. Furthermore, it was shown that, with −7 to −8% in comparison with the LPS control, the inhibition by 5-ASA, however, was only weak. Furthermore, the inhibitory effects when using higher concentrations of 5-ASA of 10 mg/ml decreased again. Overall, it can be concluded that 5-ASA has only a relatively weak anti-inflammatory effect, in particular with regard to inhibition of the production of TNF-alpha.
It was found in the context of the present invention that, surprisingly, the inhibitory effects on TNF-alpha increase with coincubation of a relevant plasma level of 5-ASA of 1 mg/ml and vitamin D in increasing, therapeutically relevant vitamin D concentrations (FIG. 5).
FIG. 5 shows that the LPS-stimulated TNF-alpha production is significantly inhibited by increasing concentrations of calcitriol (solid line). Coincubation of calcitriol with a plasma-relevant concentration of 5-ASA (1 μg/ml, dashed line) leads to a significant and also synergistic increase in TNF-alpha inhibition in comparison with the control (LPS/5-ASA), 5-ASA or vitamin D alone. The inhibitory effects are not significant for a relatively high concentration of 5-ASA (10 μg/ml) alone and increase further according to dose after coincubation of calcitriol with 5-ASA (10 μg/ml, dashed-and-dotted line). In FIG. 5, the x-axis shows the concentration of calcitriol used in ng/ml, and the y-axis shows the respective mean with standard deviation of the percentage inhibition in comparison with the TNF-alpha control (with p<0.003 vs. LPS control; *p<0.0352 vs. LPS control; **p>0.2 vs. LPS control; p<0.0001 for calcitriol+5-ASA vs. 5-ASA alone; ***p<0.022 for calcitriol+5-ASA vs. 5-ASA alone).
On the basis of FIG. 5, it is thus clear that the LPS-stimulated production of TNF-alpha is significantly inhibited by increasing concentrations of calcitriol. The coincubation of calcitriol with a plasma-relevant concentration of 5-ASA (1 mg/ml) leads furthermore to a significant and synergistic increase in inhibition of the production of TNF-alpha in comparison with the controls based on 5-ASA or calcitriol alone and also 5-ASA in combination with LPS. The inhibitory effects are furthermore not significant for a relatively high 5-ASA concentration of 10 mg/ml alone. In addition, the inhibitory effects increase further according to dose after coincubation of calcitriol with 10 mg/ml 5-ASA.
The series of experiments based on coincubation of calcitriol and 5-ASA having a concentration of 1 mg/ml shows that the combined use leads to a synergistic inhibitory action on the production of TNF-alpha. In comparison with the sole use of 5-ASA, it was possible to observe the inhibitory action over the entire therapeutic range with increasing vitamin D or calcitriol concentrations of 1 ng/ml (inhibitory action −23.1±3%), 10 ng/ml (inhibitory action −25.3±3%), 30 ng/ml (inhibitory action −27.8±3%) and 50 ng/ml (−29.4±2%). Overall, the results additionally show that the inhibition of the production of TNF-alpha also significantly rises with increasing calcitriol concentration. Thus, in the context of the present invention, an increase in the anti-inflammatory action in comparison with 5-ASA alone was, surprisingly, found for the combination of calcitriol and 5-ASA in the range of 400 to 500%.
On the basis of the stronger, anti-inflammatory action of calcifediol in comparison with calcitriol, as described according to the invention, it was furthermore possible in the context of the present invention to detect a much stronger intensification of the 5-ASA action for the combination of calcifediol and 5-ASA. This means that, for reinforcement of the relatively weak anti-inflammatory action of 5-ASA, the combination based on calcifediol and 5-ASA is even stronger and thus represents a preferred embodiment. Furthermore, this is also of relevance to the effect that the probability of developing hypercalcemia in the case of an existing situation of vitamin D deficiency can be classified as low. In addition, by using calcifediol as vitamin D receptor agonist, the vitamin D₃ receptor is not down regulated.
With this concept according to the invention of a specific combination of vitamin D and 5-ASA, it is possible in the context of the present invention to provide an improved treatment of in particular also endoluminal inflammation in the case of inflammatory bowel diseases. In the context of a treatment of this type, a positive systemic effect which brings about prophylaxis, stabilization, maintenance of remission and steroid conservation or intensification of steroid action is additionally achieved. Particularly in the case of early therapy use, the therapy concept according to the invention can thus provide an action-efficient alternative and prophylaxis of resistance development with respect to TNF-alpha inhibitors, in particular on the basis of intensification of the action of the guideline therapeutics used for the treatment of inflammatory bowel diseases. Thus, the results according to the invention are a central prerequisite for a new therapy concept for improving the often insufficient anti-inflammatory, local and systemic actions of in particular 5-ASA and budesonide for the treatment of inflammatory bowel diseases.
This action also applies to the prophylaxis and therapy of irritable bowel syndrome. In connection with the treatment of irritable bowel syndrome, the comedication of vitamin D with 5-ASA and possibly budesonide represents a new therapy form for increasing anti-inflammation.

### d) Use and Efficacy Studies

In the context of a first set of investigations, 11 patients from 32 to 65 years of age suffering from persistent asthma (GINA II) were treated with a combination therapy composed of inhaled glucocorticoid (beclomethasone, 2×200 μg/which inhalational) and inhaled long-acting beta-2 sympathomimetics (LABA, salmeterol) and also, perorally, theophylline. After one week of therapy, it was possible to achieve mild improvement of pulmonary function in 5 of the 11 test subjects. After continued long-term therapy for 12 weeks, the persistent bronchial asthma had further stabilized, and so it was possible to reduce the inhaled glucocorticoid demand by 35% in 7 of the 11 patients. In two of the treated patients, it was also possible to reduce the beta-2 agonist demand by 10%. A further group based on 9 patients from 39 to 70 years of age having an identical indication, i.e., persistent asthma (GINA II), who were similarly administered with the abovementioned combination therapy composed of inhaled glucocorticoid and inhaled long-acting beta-2 sympathomimetics and also theophylline additionally received vitamin D for one week in a daily dose of 80 μg. After one week of therapy with the aforementioned dose, it was possible to achieve a mild to moderate improvement in pulmonary function in 6 of the 9 test subjects. After continued long-term therapy for 12 weeks, the persistent bronchial asthma had stabilized insofar as it was possible to reduce the inhaled glucocorticoid need by up to 60% in 7 of the 9 patients and it was possible to even completely temporarily discontinue the inhaled glucocorticoids in 1 of the 9 patients. The therapy was well tolerated with no adverse effects. In 4 of the treated patients, it was also possible to reduce the beta-2 agonist need by up to 40%.

### e) Further Use and Efficacy Studies:

Similarly, in the context of the efficacy investigation, a dosage form according to the present invention containing vitamin D and 5-aminosalicylic acid as active ingredients was used in the treatment of Crohn's disease and ulcerative colitis in comedication in relation to systemic corticosteroids.
As comparision, a monopreparation having the same quantity of active ingredient based on 5-aminosalicylic acid as sole active substance is used.
In the context of the clinical use observations, 8 test subjects in each case (one group from 32 to 59 years of age, and the other group from 26 to 60 years of age) were treated, one group with the monopreparation based on 5-aminosalicylic acid and the other group with the above-described dosage form according to the invention.
With the dosage form according to the invention based on the combination of 5-aminosalicylic acid and vitamin D, it was possible to produce a distinctly improved clinical effect which manifests itself in that, in comparison with the group which received the monopreparation and in which the bowel movement frequency decreased by about 40%, it was possible to again distinctly improve the frequency to a total reduction of over 55%. Likewise, in comparison with the treatment with the monopreparation, it was also possible to again distinctly reduce systemic corticosteroid administration.
Overall, the present efficacy studies provide evidence for the outstanding and synergistic action of the combination mentioned according to the invention composed of, firstly, a vitamin D receptor agonist (VDA) and, secondly, a monoterpene.

### Summary:

The investigations undertaken in the context of the present invention show that the stimulation of the vitamin D receptor with the agonists calcitriol and calcifediol has an independent, anti-inflammatory action even at the lower normal limit of vitamin D of ng/ml. In particular, in comparison with the control, i.e., merely with LPS-stimulated human monocytes without incubation of calcitriol or calcifediol, calcitriol and calcifediol mediate inhibition of LPS-stimulated production of TNF-alpha of 30 and 82%, respectively.

Interactions of vitamin D or its metabolites with various, predominantly anti-inflammatory guideline therapeutics make it possible in the context of the present invention to develop specific anti-inflammatory combinations, preferably on the basis of calcifediol with at least one guideline therapeutic. In particular, these novel combinations are suitable for intensifying the guideline therapy. This effect can be attributed particularly to the suprisingly discovered, synergistically increasing anti-inflammation owing to the additional use of vitamin D metabolites. Overall, the therapy concepts according to the invention allow the treatment of different inflammatory bowel diseases and airways diseases associated with or without vitamin D deficiency.

The present invention will be described in more detail by the following aspects or subjects of the invention, and it is the case for all aspects that the described combination therapeutics and compositions do not contain in each case an ingredient and/or active ingredient from the group consisting of terpenes, monoterpenes, vasoconstrictors, alpha sympathomimetics and/or the physiologically safe salts or derivatives thereof. This is similarly the case for those compositions used according to the invention in the context of comedication and/or add-on therapy with the combination therapeutics or compositions in question:

### Aspect 1:

Vitamin D receptor agonists (VDA), in particular 25-OH D₃ (calcifediol) or analogs for the prophylaxis and therapy of anti-inflammation in the case of allergic, chronic, infectious, exacerbated local and systemic diseases, with the goal of reducing the therapy requirement or intensifying the main action of recognized guideline therapeutics irrespective of a state of vitamin D deficiency.

### Aspect 2:

Vitamin D receptor agonists (VDA), in particular 25-OH D₃ (calcifediol) or analogs preferably for the prophylactic, local, topical and inhalational therapy of upper and lower respiratory tract diseases and also for the local therapy of inflammatory bowel diseases, in particular dissolved in small intestine-soluble capsules or in the form of pellets irrespective of a state of vitamin D deficiency.

### Aspect 3:

Vitamin D receptor agonists (VDA), in particular 25-OH D₃ (calcifediol), 1,25-DOH D₃ (calcitriol) or analogs thereof, preferably in local, topical form in the case of colds, chronic obstructive pulmonary diseases and inflammatory bowel diseases for inactivating or inhibiting the propagation of viral and bacterial infectious agents, in particular with the goal of reducing the virulence, infectivity, chronically persistent inflammation response and thus the therapy requirement of antibiotics, corticosteroids and other immunosupressants irrespective of states of vitamin D deficiency.

### Aspect 4:

25-OH D₃ (calcifediol) in combination of 1,25-DOH D₃ (calcitriol) for intensifying the actions according to aspects 1 to 3 by an increase in anti-inflammation and an intensification of action by reduction of the 1,25-DOH D₃-mediated downregulation of the vitamin D₃ receptor.

### Aspect 5:

Vitamin D₃ is used as a tablet and/or small intestine-soluble capsule (2×daily 800 IU to 2000 IU), for topical nasal therapy (50-100 μg/puff, 1-2×daily) or for inhalation as suspension or powder form (250 μg/puff or 250-500 μg/powder).

### Aspect 6:

Use of 25-OH D₃ and 1,25-DOH D₃ or analogs thereof for local-nasal, inhalational or systemic therapy for the prophylaxis and therapy of viral and bacterial colds, in particular also in combination vitamin C, vitamin E and acetylsalicylic acid or another nonsteroidal anti-inflammatory drug (from the group of NSAIDs) or paracetamol for reinforcing the efficacy thereof with improvement and reduction of the progress of colds.

### Aspect 7:

Use of 25-OH D₃ or analogs, as combination therapeutic, in particular in the form of a kit as nasal spray, with topical nasal glucocorticosteroids for intensifying steroid action in upper respiratory tract diseases, in particular acute, allergic, chronic and chronically recurring, polypoid rhinitis or rhinosinusitis.

### Aspect 8:

Use of 1,25-DOH D₃, in particular of 25-OH D₃, or analogs as combination therapeutic, in particular in the form of a kit as tablet or capsule, in connection with leukotriene receptor antagonists, in particular montelukast, pranlukast, zafirlukast, 5-lipoxygenase inhibitors or antiallergics or antihistamines for intensifying the guideline therapy recommendations in the case of allergic diseases, in particular allergic asthma, or allergic rhinitis and conjunctivitis, neurodermitis and food allergies.

### Aspect 9:

Use of 1,25-DOH D₃, in particular of 25-OH D₃, or analogs thereof as combination therapeutic, in particular in the form of a kit for inhalation, in connection with ICS, LABAs, LAMAS, LABA+ICS and also LABA+ICS+LAMA, in particular with the goal of improving the therapy effect of the topical guideline therapy for lower respiratory tract diseases.

### Aspect 10:

Use of 1,25-DOH D₃, in particular of 25-OH D₃, or analogs thereof as combination therapeutic, in particular in the form of a kit as add-on therapy (+vitamin D₃ 20-30 000 IU/2 weeks), together with an anti-IgE therapy, in particular with omalizumab for inhibiting the production of IgE antibodies, preferably for protecting against allergic sensitizations with simultaneous reinforcement of action and better tolerability with a possible option to reduce the dose of costly therapies with anti-IgE.

### Aspect 11:

Use according to any of the abovementioned aspects for inhibiting or modulating COPD-associated and COPD-independent aging processes, in particular with the goal of reducing disease incidence and severity and of improving life expectancy and quality of life and of delaying the entire aging process.

### Aspect 12:

Use of 1,25-DOH D₃, in particular of 25-OH D₃, or analogs thereof as combination therapeutic, in particular in the form of a kit as small intestine-soluble capsule, in connection with 5-aminosalicylic acid (vitamin D₃+5-ASA), a glucocorticosteroid, in particular budesonide (vitamin D₃+budesonide), and a cytokine inhibitor or receptor antagonist, or other biologics for intensifying the guideline therapy of inflammatory bowel diseases (IBD) with the small and large intestine being affected, in particular Crohn's disease (CD) and ulcerative colitis (CU), hepatic autoimmune diseases, such as in the case of primary biliary cirrhosis, for the prophylaxis and increase of steroid action in the case of IBD.

### Aspect 13:

Prophylaxis of inflammatory bowel diseases by antioxidative agents, in particular a combination of vitamin D₃, vitamin C, and/or vitamin E with budesonide in small intestine-soluble capsules and in the form of food additive.

### Aspect 14:

Use of 1,25-DOH D₃, in particular of 25-OH D₃, or analogs thereof as combination therapeutic, in particular in the form of a kit as small intestine-soluble capsule, in connection with 5-ASA or derivatives, budesonide or other glucocorticosteroids, for local, systemic and synergistic reinforcement of the guideline therapy of the intestinal diseases mentioned under aspect 12 with a specific focus on clinical superiority by reduction of acute, infectious, allergically or chronically related relapse incidences with improvement of therapy refractoriness through a new cause-related approach for combined treatment of local and systemic inflammation in the case of IBD.

1-64. (canceled)

65. A pharmaceutical combination therapeutic for use in the prophylactic and therapeutic treatment of (A) airways diseases and (B) inflammatory diseases of the intestine,
wherein the combination therapeutic comprises, each in pharmaceutically effective quantities:
(a) at least one vitamin D receptor agonist (VDA); and
(b) at least one further active ingredient,
(b1) the further active ingredient being at least one topical corticosteroid in the case of the prophylactic and therapeutic treatment of (A) airways diseases; or
(b2) the further active ingredient(s) being 5-aminosalicylic acid and its physiologically compatible salts or esters and optionally at least one systemic corticosteroid in the case of the prophylactic and therapeutic treatment of (B) inflammatory diseases of the intestine;
however, with the proviso that the combination therapeutic does not contain an ingredient or active ingredient selected from the group consisting of terpenes, monoterpenes, vasoconstrictors, alpha sympathomimetics and their physiologically compatible salts and derivatives.

66. The combination therapeutic as claimed in claim 65, wherein (a) the vitamin D receptor agonist (VDA) is selected from the group consisting of compounds of the vitamin D group.

67. The combination therapeutic as claimed in claim 65, wherein (a) the vitamin D receptor agonist (VDA) is selected from the group consisting of vitamin D₃ (cholecalciferol), vitamin D₂ (ergocalciferol), 25-hydroxyvitamin D₃ (calcifediol), 1,25-dihydroxyvitamin D₃ (calcitriol), physiological vitamin D₃ precursors, physiological vitamin D₃ metabolites, synthetic vitamin D₃ analogs, tacalcitol and combinations thereof.

68. The combination therapeutic as claimed in claim 65, wherein (a) the vitamin D receptor agonist (VDA) is a combination of 25-hydroxyvitamin D₃ (calcifediol) and 1,25-dihydroxyvitamin D₃ (calcitriol).

69. The combination therapeutic as claimed in claim 65, wherein the combination therapeutic comprises (a) the vitamin D receptor agonist (VDA) in a relative quantity in the range of 0.0001% by weight to 5% by weight, based on the combination therapeutic.

70. The combination therapeutic as claimed in claim 65, wherein the combination therapeutic comprises (a) the vitamin D receptor agonist (VDA in an absolute quantity in the range of 0.025 μg to 400 μg, based on one dosage or administration unit of the combination therapeutic.

71. The combination therapeutic as claimed in claim 65, wherein the combination therapeutic further comprises at least one physiologically compatible excipient.

72. The combination therapeutic as claimed in claim 65, wherein (b1) the topical corticosteroid is a glucocorticoid selected from the group consisting of beclomethasone, mometasone, budesonide, flunisolide, fluticasone, triamcinolone and their physiologically compatible derivatives, salts and esters and mixtures and combinations of the aforementioned compounds.

73. The combination therapeutic as claimed in claim 65, wherein the combination therapeutic comprises (b1) the topical corticosteroid in a relative quantity in the range of 0.001% by weight to 10% by weight, based on the combination therapeutic.

74. The combination therapeutic as claimed in claim 65, wherein (b2) 5-aminosalicylic acid is used in the form of its physiologically compatible alkali and alkaline earth metal salts.

75. The combination therapeutic as claimed in claim 65, wherein the combination therapeutic comprises (b2) 5-aminosalicylic acid in a relative quantity in the range of 0.001% by weight to 50% by weight, based on the combination therapeutic.

76. The combination therapeutic as claimed in claim 65, wherein the combination therapeutic comprises at least one further pharmacologically effective ingredient selected from the group consisting of anti-inflammatory drugs, nonsteroidal anti-inflammatory drugs, acetylsalicylic acid, paracetamol; beta-sympathomimetics, beta-2-sympathomimetics; parasympatholytics; vagolytics; anticholinergics; and mixtures and combinations of the aforementioned compounds.

77. The combination therapeutic as claimed in claim 65, wherein (b2) the systemic corticosteroid is a glucocorticoid selected from the group consisting of dexamethasone, prednisone, prednisolone, betamethasone, rimexolone, paramethasone, hydrocortisone and the physiologically compatible derivatives, salts and esters thereof and mixtures and combinations of the aforementioned compounds.

78. The combination therapeutic as claimed in claim 65, wherein the combination therapeutic comprises (b2) the systemic corticosteroid in a relative quantity in the range of 0.001% by weight to 10% by weight, based on the combination therapeutic.

79. The combination therapeutic as claimed in claim 65, wherein the combination therapeutic comprises at least one further vitamin selected from the group consisting of vitamin A, vitamin C and vitamin E.

80. The combination therapeutic as claimed in claim 65,
wherein the combination therapeutic is delivered in daily doses of (a) 10 μg to 1000 μg of the vitamin D receptor agonist (VDA);
wherein the combination therapeutic is delivered in daily doses of (b1) 50 to 1000 μg of the topical corticosteroid;
wherein the combination therapeutic is delivered in daily doses of (b2) 50 to 10 000 mg of 5-aminosalicylic acid;
wherein the combination therapeutic is delivered in daily doses of (b2) 50 to 1500 μg of the systemic corticosteroid.

81. The combination therapeutic as claimed in claim 65, wherein the combination therapeutic comprises, firstly, (a) the vitamin D receptor agonist (VDA) and, secondly, (b) the further active ingredient in a joint composition or, alternatively, in separate compositions in the form of a kit-of-parts.

82. A method of treating a patient suffering from a disease selected from (A) airways diseases and (B) inflammatory diseases of the intestine, the method comprising administering to the patient a pharmaceutically effective amount of the pharmaceutical combination therapeutic of claim 65.
